(19) United States
(12) Patent Application Publication
(10) Pub. No.: US 2024/0142403 A1
(43) Pub. Date: May 2, 2024

(54) Methods of Breath-Based Toxin Exposure Assessment, and Systems for Practicing the Same

(71) Applicant: Diagnose Early, Inc., Menlo Park, CA (US)

(72) Inventors: Chris Wheeler, Menlo Park, CA (US); Karl-Magnus Larsson, Menlo Park, CA (US); Kevin Bundy, Menlo Park, CA (US); Luke Clauson, Menlo Park, CA (US)

(21) Appl. No.: 18/382,736

(22) Filed: Oct. 23, 2023

Related U.S. Application Data
(60) Provisional application No. 63/420,375, filed on Oct. 28, 2022.
(60) Provisional application No. 63/529,245, filed on Jul. 27, 2023.

(51) Int. Cl. G01N 27/623; G16H 50/20

(52) U.S. Cl. CPC G01N 27/623; G16H 50/20; G01N 2800/40

(57) ABSTRACT

Methods of assaying a breath sample from a subject for the presence of one or more toxins or toxin associated compounds are provided. In some embodiments, the one or more toxins or toxin associated compounds includes trichloroethylene (TCE) and/or one or more metabolites thereof. Embodiments of the methods further include providing a toxin exposure assessment that can be used to determine if the subject has been exposed to a toxin and to monitor changes in toxin exposure over time in the subject. In certain embodiments, a machine learning model may be used to determine if a subject has been exposed to one or more toxins. In some embodiments, the machine learning model may be trained by: analyzing breath samples from a plurality of subjects with a secondary electrospray ionization-high-resolution mass spectrometry analyzer to generate breath assay data; obtaining toxin exposure data for each subject; training a machine learning model to identify a relationship between the breath samples and toxin exposure using the breath assay data and the obtained toxin exposure data; and applying the trained machine learning model to breath assay data, different from the breath assay data used to train the model, to generate an exposure assessment regarding potential toxin exposure for a subject or subjects. In some embodiments, a health evaluation is generated for the subject using the toxin exposure assessment. Also provided are systems for use in practicing methods of the invention.

### CROSS-REFERENCE TO RELATED APPLICATIONS

Pursuant to 35 U.S.C. § 119 (e), this application claims priority to the filing dates of U.S. Provisional Patent Application Ser. No. 63/420,375, filed on Oct. 28, 2022 and U.S. Provisional Patent Application Ser. No. 63/529,245, filed on Jul. 27, 2023, the disclosures of which applications are herein incorporated by reference.

### INTRODUCTION

Many chemicals, man-made and otherwise, can be highly toxic when absorbed into the body through skin contact, ingestion or respiration. The modern world has exposed us to more and more of these chemicals. Although we still do not fully understand the impact of exposure to these chemicals, many of them are proven or suspected of being carcinogens.

Certain industries and careers lead to an especially high risk of exposure to these toxins. Famously, huge numbers of asbestos miners suffered from mesothelioma, a previously rare form of cancer. Recently, firefighting was classed as carcinogenic due to the high rate of exposure firefighters have to many carcinogens through, e.g., breathing in smoke, among other things. The International Association of Firefighters (IAFF) has compiled a list of high-priority carcinogenic agents to raise awareness and reduce exposure.

Toxin exposure assessments have typically involved assaying biological samples, such as blood or urine, using time consuming and costly protocols.

### SUMMARY

The inventors have realized that methods of rapidly determining toxin exposure from non-invasively obtained samples, and specifically breath samples, are needed. Embodiments of the invention meet this need. Methods of assaying a breath sample from a subject for the presence of one or more toxins or toxin associated compounds are provided. In some embodiments, the one or more toxins or toxin associated compounds include trichloroethylene (TCE) and/or one or more metabolites thereof.

Embodiments of the methods further include providing a toxin exposure assessment that can be used to determine if the subject has been exposed to a toxin and to monitor changes in toxin exposure over time in the subject. In certain embodiments, a machine learning model may be used to determine if a subject has been exposed to one or more toxins. In some embodiments, the machine learning model may be trained by: analyzing breath samples from a plurality of subjects with a secondary electrospray ionization-high-resolution mass spectrometry analyzer to generate breath assay data; obtaining toxin exposure data for each subject; training a machine learning model to identify a relationship between the breath samples and toxin exposure using the breath assay data and the obtained toxin exposure data; and applying the trained machine learning model to breath assay data, different from the breath assay data used to train the model, to generate an exposure assessment regarding potential toxin exposure for a subject or subjects. In some embodiments, a health evaluation is generated for the subject using the toxin exposure assessment. Also provided are systems for use in practicing methods of the invention.

### BRIEF DESCRIPTION OF THE FIGURES

FIG. 1 provides a depiction of an overview of the results obtained from a breath sample assay in accordance with an embodiment of the invention.

FIG. 2 provides a flow diagram depicting a method for dynamically adjusting breath collection automatically based on real-time feedback in accordance with an embodiment of the invention.

FIGS. 3A-3B illustrate selected ion monitoring (SIM) automatically performed based on real-time feedback in accordance with an embodiment of the invention.

FIG. 4 provides a depiction of a health evaluation obtained at least in part from a toxin exposure assessment in accordance with an embodiment of the invention.

FIG. 5 illustrates various metabolic profiles of a health report obtained at least in part from a toxin exposure assessment generated from a breath sample assay in accordance with an embodiment of invention.

FIGS. 6A-6B provide a depiction of a toxin exposure assessment obtained at least in part from a breath sample assay in accordance with an embodiment of invention.

FIG. 7 provides a flow diagram depicting a method for training a machine learning model using generated breath assay data and toxin exposure data in accordance with an embodiment of the invention.

### DETAILED DESCRIPTION

Methods of assaying a breath sample from a subject for the presence of one or more toxins or toxin associated compounds are provided. In some embodiments, the one or more toxins or toxin associated compounds includes trichloroethylene (TCE) and/or one or more metabolites thereof. Embodiments of the methods further include providing a toxin exposure assessment that can be used to determine if the subject has been exposed to a toxin and to monitor changes in toxin exposure over time in the subject. In certain embodiments, a machine learning model may be used to determine if a subject has been exposed to one or more toxins. In some embodiments, the machine learning model may be trained by: analyzing breath samples from a plurality of subjects with a secondary electrospray ionization-high-resolution mass spectrometry analyzer to generate breath assay data; obtaining toxin exposure data for each subject; training a machine learning model to identify a relationship between the breath samples and toxin exposure using the breath assay data and the obtained toxin exposure data; and applying the trained machine learning model to breath assay data, different from the breath assay data used to train the model, to generate an exposure assessment regarding potential toxin exposure for a subject or subjects. In some embodiments, a health evaluation is generated for the subject using the toxin exposure assessment. Also provided are systems for use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of assaying a breath sample from a subject for the presence of one or more toxins or toxin associated compounds. In some embodiments, the one or more toxins or toxin associated compounds includes trichloroethylene (TCE) and/or one or more metabolites thereof. Embodiments of the methods further include providing a toxin exposure assessment that can be used to determine if the subject has been exposed to a toxin and to monitor changes in toxin exposure over time in the subject. In certain embodiments, a machine learning model may be used to determine if a subject has been exposed to one or more toxins. In some embodiments, the machine learning model may be trained by: analyzing breath samples from a plurality of subjects with a secondary electrospray ionization-high-resolution mass spectrometry analyzer to generate breath assay data; obtaining toxin exposure data for each subject; training a machine learning model to identify a relationship between the breath samples and toxin exposure using the breath assay data and the obtained toxin exposure data; and applying the trained machine learning model to breath assay data, different from the breath assay data used to train the model, to generate an exposure assessment regarding potential toxin exposure for a subject or subjects. In some embodiments, a health evaluation is generated for and provided to the subject using the toxin exposure assessment and, e.g., one or more health records obtained for the subject.

Additional aspects of the invention may be found in greater detail below. The methods allow for rapidly determining toxin exposure from non-invasively obtained samples, such as breath samples, which can provide for a number of advantages including, but not limited to, improvements in turnaround time, usability, cost, and performance.

### Analyzing the Breath Sample

As described above, embodiments of the methods include analyzing breath samples from a plurality of subjects with a breath analyzer. The breath sample of the subject or subjects that is analyzed (i.e., assayed) may vary, and may be made up of 1 or more breaths, where in some instances the number of breaths ranges from 1 to 25, such as 1 to 20, including 1 to 15, e.g., 1 to 10, including 1 to 5 exhaled breaths. In some instances, the period of time between each exhaled breath received for the breath assay may vary, where in some instances the time between each received exhaled breath ranges from 1 to 180 seconds, such as 10 to 120, including 15 to 100, e.g., 20 to 90, including 20 to 60 seconds. In other embodiments, each exhaled breath of the breath sample may be received consecutively with respect to the previously received exhaled breath.

The breath sample may be a gaseous breath sample or an exhaled breath condensate (EBC) of the breath sample. In embodiments wherein the breath sample is an EBC, the EBC may be collected by having the subject exhale into a container, cooling the container, then collecting the EBC on the inside walls of the cooled container. The container may be cooled by, e.g., chilling the container in a freezer or refrigerator, with dry ice, or using liquid nitrogen. In some embodiments, the EBC may be stored for a period of time before assaying. In some instances, the EBC, is stored for a period of time such as 24 hours or more, or 48 hours or more, or 72 hours or more, or 4 days or more, or 5 days or more, or 6 days or more, or 1 week or more, or 2 weeks or more, or 3 weeks or more, or 4 weeks or more, or 1 month or more. In embodiments where the breath sample is a condensate, methods may include aerosolization of the condensate prior to assaying using, e.g., a nebulizer.

Embodiments of the method may further include shipping the breath sample (e.g., EBC) to a remote location for assaying. A “remote location,” is a location other than the location at which the breath sample is collected. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or one hundred miles or more apart.

Breath analyzers, in accordance with embodiments of the methods, may vary. In some embodiments, the analyzer includes a Raman spectroscopy analyzer, a breathalyzer, an optical absorbance sensing analyzer, a gas chromatography analyzer, electronic sensing using an electronic nose, a nuclear magnetic resonance spectroscopy analyzer, or a mass spectrometry analyzer. In some embodiments, the breath analyzer includes a mass spectrometry analyzer such as, e.g., a high-resolution mass spectrometry (HRMS) analyzer. In embodiments where the breath analyzer includes a mass spectrometry analyzer (i.e., the analyzer is configured to perform mass spectrometry), the mass spectrometry method/technique employed by the analyzer may vary and the analyzer may be coupled with or include (e.g., may be configured to perform) one or more of: ion mobility spectrometry (IMS), gas chromatography (GC), liquid chromatography (LC), differential mobility spectrometry (DMS), field asymmetric ion mobility spectrometry (FAIMS), a selective-ion flow tube (i.e., SIFT-MS), a proton-transfer-reaction (i.e., PTR-MS), time-of-flight mass spectrometry (TOF-MS) etc. For example, the mass spectrometry analyzer may perform IMS-mass spectrometry (IMS-MS), GC-mass spectrometry (GC-MS), LC-mass spectrometry (LC-MS), etc. In some embodiments, tandem mass spectrometry may be performed using, e.g., two or more mass spectrometry analyzers.

In embodiments where the breath analyzer includes a mass spectrometry analyzer, the ionization method/technique employed by the analyzer may vary and may include matrix-assisted laser desorption/ionization (MALDI), atmospheric pressure chemical ionization (APCI), atmospheric pressure photoionization (APPI), electrospray ionization (ESI), secondary electrospray ionization (SESI), etc. In some embodiments, the ionization technique employed is a soft ionization technique. In these instances, the mass spectrometry analyzer may be configured to perform SESI such as, e.g., SESI-HRMS or SESI-TOF-HRMS. In embodiments where the ionization method/technique employed by the mass spectrometry analyzer is SESI, the breath sample may be a gaseous breath sample (e.g., collected directly from the subject or aerosolized after being collected as an EBC). In embodiments where the ionization method/technique is SESI, the mass spectrometry analyzer may include a SUPER SESI™ (e.g., SUPER SESI™-HRMS) device.

As described above, the mass spectrometry analyzer may be configured to perform SESI mass spectrometry (e.g., SESI-HRMS). The SESI mass spectrometry may be run in positive-ion mode (i.e., wherein ionization occurs through protonation, or positive ions enter the mass spectrometer) or negative-ion mode (i.e., wherein ionization occurs through deprotonation, or negative ions enter the mass spectrometer). In some embodiments, the SESI mass spectrometry analyzer is run in negative-ion mode. The ionization agent of the SESI mass spectrometry analyzer may vary and may include a fluid such as, e.g., a liquid. By ionization agent (or, i.e., charging/ionizing agent) is meant the one or more chemical elements or compounds responsible for creating (i.e., producing) primary ions or, e.g., the ion source from which primary ions are generated. In these instances, the primary ions generated or produced may then interact with particles (e.g., toxins or toxin associated compounds) of the breath sample in gas phase (e.g., vapor particles) in order to produce or generate secondary ions that are detected by the mass spectrometry analyzer. In some embodiments, the ionization agent used to produce an electrospray of primary ions may include a liquid solution. The liquid solution may include any number of different solvents (e.g., protic solvents, aprotic solvents etc.) or solutes/additives (e.g., charging reagents), as well as combinations thereof, as are known in the art. In some embodiments, the solution may include, but is not limited to, a solvent composed of or including water or methanol. In some instances, the solution may include a solvent primarily composed of water. In these cases, the solution may include a solvent primarily composed or consisting of high purity grade water. In some embodiments, the solution may include, but is not limited to, a solute including a charging reagent such as, e.g., an acid or a base. In some instances, the solution may include a base or deprotonation agent such as, e.g., a hydroxide (e.g., ammonia hydroxide) or a hydride. In some instances, the solution may include an acid such as, e.g., formic acid, carbonic acid, acetic acid, nitric acid, etc. In some cases, the solution may include formic acid or carbonic acid. In these instances, the solution may include a solvent primarily composed of water and formic acid or carbonic acid. For example, in embodiments where the ionization agent includes formic acid, the formic acid may be diluted in water, such as diluted to achieve a ratio ranging from 0.01-1.0% volume over volume (v/v) of formic acid to water, such as 0.05-0.5% v/v of formic acid to water, or 0.1-0.2% v/v of formic acid to water.

As discussed above, the ionization agent of the mass spectrometry analyzer may include any number of different solvents and solutes in order to generate an electrospray of primary ions. The ionization agent (e.g., ionization/charging solution) may vary depending on any number of different factors or circumstances. In some embodiments, the ionization agent may vary depending on the polarity or mode (e.g., negative-ion or positive-ion) in which the SESI is being run or, i.e., the ionization agent may be configured or adapted specifically to produce positive and/or negative primary ions. In some cases, the ionization agent may vary depending on the mode of delivery of the breath sample into the mass spectrometry analyzer (e.g., breath delivered directly from the subject or aerosolized after being collected as an EBC). In some instances, the ionization agent may depend on a specific species/compound of interest that may be present in the breath sample or, i.e., the ionization agent may be selected in order to ionize specific compounds or narrow the number of compounds that are ionized. In some embodiments, the ionization agent may be configured to facilitate fragmentation, e.g., according to any of the methods or techniques discussed below.

As discussed above, mass spectrometry methods and techniques employed in accordance with embodiments of the methods may vary. In some embodiments, mass spectrometry techniques that may be employed include, but are not limited to, those disclosed in U.S. Pat. No. 11,075,068 and the patent documents cited therein, which methods are incorporated herein by reference; and Singh, K. D., Tancev, G., Decrue, F. et al. Standardization procedures for real-time breath analysis by secondary electrospray ionization high-resolution mass spectrometry. Anal Bioanal Chem 411, 4883-4898 (2019). https://doi.org/10.1007/s00216-019-01764-8. In some embodiments, the mass spectrometry analyzer may be a Thermo Scientific high-resolution mass spectrometer (e.g., Thermo Scientific Exactive™ or Q-Exactive™) or a SciEX high-resolution mass spectrometer (e.g., a TripleTOF® mass spectrometer system).

In some embodiments, the breath sample is assayed in real time with respect to the subject providing the breath sample. Assaying the breath sample in real time with respect to the subject providing the breath sample may, e.g., minimize any chemical changes taking place which may impact the results of the breath sample assay. In these embodiments, compounds that are exhaled from deeper in the lungs may be detected relatively later in the assay. In some embodiments, the time of detection of a compound in the breath sample assay is used to identify and validate the detection of the compound (e.g., a toxin) or provide other information regarding, e.g., the fingerprint of a toxin or toxin source or the pharmacokinetics of a toxin.

In some embodiments, real-time feedback of the measurements of the mass spectrometry analyzer may be generated and used to enhance the accuracy of relevant measurements. By relevant measurement is meant a mass-to-charge ratio (m/z) measurement of a feature of interest. In some embodiments, the feature of interest may be a toxin of interest (e.g., the m/z of the toxin of interest or a metabolite thereof). In some embodiments, the feature of interest may be one or more m/z measurements of a toxin or toxin source fingerprint. By fingerprint is meant a unique set of identified (e.g., as unique compounds or metabolites thereof) and/or unidentified m/z peaks or measurements and the context of the m/z peaks or measurements (e.g., the relative intensities of the m/z peaks, the temporal position of the m/z peaks in a breath, and/or any other context determined to be significant by a machine learning model during training whether known or unknown as discussed in greater detail below) that are unique to a specific subject, sample type, compound and/or circumstance. For example, a subject's breath may have a specific fingerprint, a toxin may have a specific fingerprint such that it is able to be identified in a subject's breath, a toxin source may have a specific fingerprint such that exposure of a subject to the toxin source be determined using the subject's breath, etc.

In some cases, a toxin or toxin source fingerprint may include a direct measurement of a toxin and/or a metabolite of a toxin and one or more measurements of other compounds that are not toxins or known metabolites thereof (including, e.g., measurements of unidentified compounds). In other instances, a toxin or toxin source fingerprint may include only measurements of compounds that are not toxins or known metabolites thereof (i.e., identified compounds that are not toxins or metabolites thereof and unidentified compounds [i.e., unidentified measurements]). Compounds of a toxin or toxin source fingerprint that are not toxins or known metabolites thereof may include compounds typically found with a toxin or toxin source (i.e., compounds that the subject is exposed to alongside the toxin or toxin source) or may be associated with the subject's body (i.e., cells of the subject's body) reacting to the toxin or toxin source. In some instances, the reason a compound of a toxin or toxin source fingerprint that is not a toxin, or a known metabolite thereof, is included in the fingerprint is unknown.

In some cases, the fingerprint may include the abundance (e.g., concentration) of a unique set of metabolites or other compounds in relation to each other or in relation to other compounds found in the subject's breath (i.e., the relative abundance of the set of metabolites or other compounds or combinations thereof) determined using identified m/z peaks. In some instances, the fingerprint may include a temporal component. For example, the relative intensity of a set of m/z peaks or measurements of a fingerprint may change with the time of detection (e.g., as air is exhaled from deeper portions of the lungs). In some embodiments, the fingerprint may be generated by a machine learning model as discussed in greater detail below. In these instances, the real-time measurements may be fed to the trained machine learning model in order to generate features of interest (i.e., relevant measurements) for which accuracy may be enhanced, as discussed in greater detail below. In some instances, the mass spectrometry analyzer is dynamically adjusted in real-time based on real-time measurement feedback provided, e.g., for each breath assayed from a subject.

In some embodiments, selected ion monitoring (SIM) may be performed in order to enhance the accuracy of relevant measurements. In these instances, measurements of a subject's breath generated by the mass spectrometer may be analyzed in real-time in order to search for toxins and toxin or toxin source fingerprints of interest. If evidence of a toxin and/or fingerprint is found, the mass spectrometry analyzer may be configured to only measure and/or transmit one or more m/z values of select features of interest (or, e.g., limited ranges of m/z values containing selected features) in a subsequent breath sample provided by the subject. In these instances, the mass spectrometry analyzer may be configured to measure the select features (or, e.g., select range of m/z values containing features) with enhanced sensitivity and accuracy, i.e., when compared with the measurements taken before SIM. For example, by limiting the range of detected m/z values, the mass spectrometry analyzer may boost or amplify the signal of selected features of interest. In some cases, the SIM may be dynamic within a single breath and, e.g., the selected features of interest may change throughout a single breath. For example, in embodiments where an identified toxin or toxin source fingerprint of interest is characterized by different m/z measurements (including, e.g., m/z measurements associated with identified compounds) as air is exhaled from deeper portions of the lungs, the SIM may change to monitor different m/z ranges as the time of detection within a single breath changes.

In some embodiments, SIM is performed automatically. For example, mass spectrometry measurements may be transmitted directly to a processor configured to search for compounds (e.g., toxins) and fingerprints of interest. The processor may then configure the mass spectrometry analyzer to limit detection to, and amplify the signal of, one or more select features (e.g., of toxins or toxic compound or source fingerprints of interest) for which evidence is found thereof. In some instances, the processor may be configured to automatically perform SIM using a trained machine learning model, as discussed in greater detail below.

As discussed above, real-time feedback of the mass spectrometry analyzer may be generated and used to enhance the accuracy of relevant measurements. In some embodiments, fragmentation may be performed in order to enhance the accuracy of relevant features. In some instances, fragmentation is performed on all breath samples using, e.g., tandem mass spectrometry. In other cases, fragmentation may be performed based on real-time feedback as discussed above. For example, if evidence of a toxin and/or toxic compound or source fingerprint of interest is found, the mass spectrometry analyzer may be configured to perform a fragmentation run on the toxin of interest or compounds of the toxin or toxin source fingerprint of interest. Fragmentation may vary depending on the compound or fingerprint of interest and may include, but is not limited to, collision-induced dissociation (CID), surface-induced dissociation (SID), laser induced dissociation, electron-capture dissociation (ECD), electron-transfer dissociation (ETD), negative electron-transfer dissociation (NETD), electron-detachment dissociation (EDD), photodissociation (e.g., infrared multiphoton dissociation (IRMPD) or blackbody infrared radiative dissociation (BIRD)), higher-energy C-trap dissociation (HCD), EISA, and/or charge remote fragmentation.

In some embodiments, fragmentation is performed automatically. For example, mass spectrometry measurements may be transmitted directly to a processor configured to search for compounds and fingerprints of interest. The processor may then configure the mass spectrometry analyzer to perform fragmentation of the compound of interest or compounds of the fingerprint of interest for which evidence is found thereof. In some instances, the processor may be configured to automatically perform fragmentation using a trained machine learning model, as discussed in greater detail below. In some cases the processor may be configured to automatically perform SIM and fragmentation. For example, the processor may perform SIM (e.g., as discussed above) to amplify the signal of m/z measurements pertaining to toxins and toxin or toxin source fingerprints of interest for which evidence is found thereof after receiving measurements pertaining to a first breath or group of breaths provided by a subject. If the SIM further verifies the presence of the identified toxin(s) and/or toxin or toxin source fingerprint(s) of interest, the processor may then configure the mass spectrometry analyzer to perform fragmentation of the toxin of interest or compounds of the toxin or toxin source fingerprint of interest in order to confirm the presence of the identified toxin(s) and/or toxin or toxin source fingerprint(s) of interest in the subject's breath.

As discussed above, real-time feedback of the mass spectrometry analyzer may be generated and used to enhance the accuracy of relevant measurements using, e.g., fragmentation or SIM. In some embodiments, ionization agent (e.g., ionization fluid or liquid) switching may be performed in order to enhance the accuracy of relevant features or in order to determine if certain features are present in a breath sample. As described above, the ionization agent (e.g., ionization/charging fluid) may vary depending on any number of different factors or circumstances. In some cases, the mass spectrometry analyzer may be configured to switch between multiple ionization agents such as, e.g., two or more ionization agents, or three or more ionization agents, or five or more, or ten or more. The ionization agent may be changed or switched one or more times within a single breath such as, e.g., two or more times within a single breath, or three or more times, or five or more. In some embodiments, the mass spectrometry analyzer may switch from a first ionization agent to another in order to enhance the accuracy of relevant measurements (e.g., pertaining to features of interest). In some embodiments, the mass spectrometry analyzer may switch from a first ionization agent to another in order to measure a compound not detectable using the first ionization agent.

In some embodiments, ionization agent (e.g., ionization fluid or liquid) switching is performed automatically. In these instances, the automatic ionization agent switching may be dynamic or uniform/periodic. In some embodiments, automatic ionization agent switching may be dynamic. For example, mass spectrometry measurements may be transmitted directly to a processor configured to search for compounds and fingerprints of interest. The processor may then configure the mass spectrometry analyzer to switch to an ionization agent with a relatively high (compared to, e.g., the previous ionization agent or a different compound with a similar m/z ratio) ionization efficiency for the compound of interest or compounds of the fingerprint of interest for which evidence is found thereof. In some embodiments, the processor may configure the mass spectrometry analyzer to switch to an ionization agent that ionizes a narrower number of compounds (compared to, e.g., the previously used ionization agent) such that two compounds with similar m/z ratios can be differentiated and the presence of the compound of interest or compounds of the fingerprint of interest for which evidence is found thereof can be verified.

In embodiments where the mass spectrometry analyzer is configured to switch between ionization agents that ionize, or allow the detection of, different compounds (such that, e.g., each ionization agent allows detection of a compound not enabled by the other agent) automatic ionization agent switching may be performed dynamically or uniformly (i.e., periodically). For example, in some embodiments the mass spectrometry analyzer switches between a first ionization agent and a second ionization agent at a regular and unchanging/constant interval (i.e., periodically) such that toxins ionized by each agent are simultaneously detectable in a single breath or breath sample or, e.g., a toxic compound or source fingerprint generated using both ionization agents (and generated, e.g., using a machine learning model as discussed in greater detail below) is detectable. In another example, the processor may select a set number of toxins and/or toxic compound or toxin source fingerprints of interest that are the most likely to be present in the subject's breath (or, e.g., may select all the toxins or toxic compound/toxin source fingerprints of interest that meet a predetermined threshold of probability of being in the subject's breath) from measurements of a first breath or group of breaths provided by the subject using a first ionization agent. The processor may then switch from the first ionization agent to a second ionization agent in order to verify the presence of the selected toxin or toxin source fingerprints of interest. In some cases the processor may be configured to automatically perform SIM, fragmentation, and ionization agent switching processes simultaneously as needed.

As discussed above, the mass spectrometry analyzer (e.g., SESI-HRMS) and, e.g., the ionization agent thereof, may be configured to run analysis of a breath sample in positive-ion mode or negative-ion mode. In some embodiments, positive-ion mode and negative-ion mode may be run on a single breath sample such as, e.g., a single breath of a breath sample. In some embodiments, the mass spectrometry analyzer may be configured to rapidly switch between polarities (i.e., from negative-ion mode to positive-ion mode and vice versa). In these instances, the polarity switching time of the mass spectrometry analyzer may be sufficiently short such that several measurements may be taken in positive-ion mode and negative-ion mode for, e.g., each breath of a breath sample. For example, the polarity switching time of the mass spectrometry analyzer may be 500 milliseconds (msec) or less, such as 200 msec or less, or 100 msec or less, or 50 msec or less, or 20 msec or less, or 10 msec or less, or 5 msec or less, or 1 msec or less. In some embodiments, the mass spectrometry analyzer may switch polarities in order to enhance the accuracy of relevant measurements. In some embodiments, the mass spectrometry analyzer may switch polarities in order to measure a compound not detectable using the other polarity.

In some embodiments, polarity switching is performed automatically. In these instances, the automatic polarity switching may be dynamic or uniform/periodic. In some embodiments, automatic ionization agent switching may be dynamic. For example, mass spectrometry measurements may be transmitted directly to a processor configured to search for compounds and fingerprints of interest. The processor may then configure the mass spectrometry analyzer to switch to a polarity with a relatively high (compared to, e.g., the other polarity or a different compound with a similar m/z ratio) ionization efficiency for the compound of interest or compounds of the fingerprint of interest for which evidence is found thereof. In some embodiments, the processor may configure the mass spectrometry analyzer to switch to a polarity that ionizes a narrower number of compounds (compared to, e.g., the other polarity) such that two compounds with similar m/z ratios can be differentiated and the presence of the compound of interest or compounds of the fingerprint of interest for which evidence is found thereof can be verified. In other embodiments, automatic polarity switching may be uniform/periodic. In these instances, the mass spectrometry analyzer may be configured to switch polarities at a regular and unchanging interval (i.e., periodically) such that toxins detectable in either polarity (i.e., negative-ion mode or positive-ion mode) may be detectable in a single breath or breath sample or, e.g., a toxic compound or toxin source fingerprint generated using both polarities (and generated, e.g., using a machine learning model as discussed in greater detail below) is detectable. In some cases the processor may be configured to automatically perform SIM, fragmentation, ionization agent switching, and polarity switching processes simultaneously as needed.

In some instances, one or more analyzers (e.g., as described above) may be used to further verify the presence of the identified toxin(s) and/or toxin or toxin source fingerprint(s) of interest. For example, after the method for dynamically adjusting breath collection automatically based on real-time feedback (e.g., as described above) is run, a further breath sample may be collected and analyzed using gas chromatography (GC) or liquid chromatography (LC) techniques, such as GC-MS or LC-MS. In some cases, the GC-MS or LC-MS may be coupled with SESI-HRMS including, e.g., in tandem with the SESI-HRMS.

In some embodiments, real-time feedback of measurements of the mass spectrometry analyzer may be generated and used to monitor data quality. In some embodiments, real-time feedback of the mass spectrometry analyzer may be automatically monitored in order to determine if the breath sample (i.e., or individual breaths thereof) is of a sufficient quality. By sufficient quality is meant capable of producing accurate breath assay results. In some embodiments, data quality may be monitored using a machine learning model as discussed in greater detail below. For example, real-time measurements may be fed to a trained machine learning model in order to determine if the measurements of an individual breath are of sufficient quality. In some embodiments, the subject may be prompted to provide an additional breath or additional breaths if a breath sample (i.e., or individual breaths thereof) is not of sufficient quality. In some embodiments, a technician or operator may monitor real-time feedback of the mass spectrometry analyzer in order to determine if the breath sample is of a sufficient quality or if one or more settings of the mass spectrometry analyzer should be adjusted.

While the methods of the invention may be employed on a variety of subjects, in some instances, the subject is a human. In some instances, the human is a protective service professional, a healthcare professional, a construction professional, a production professional, or a military professional, e.g., as is further detailed at: https://www.bls.gov/soc/2018/major_groups.htm. Of interest in certain embodiments is where the human is a protective service professional, such as a firefighter. In some instances, the methods of the invention may be employed on a subject wherein there is evidence the subject has been exposed to a particular toxin or toxin source.

In some embodiments, the plurality of subjects may include two or more subjects. In some instances, the plurality of subjects may include ten or more subjects, such as twenty or more, or fifty or more, or one hundred or more, or two hundred or more, or five hundred or more, or one thousand or more, or five thousand or more, or ten thousand or more, or one hundred thousand or more. The plurality of subjects may include the subjects of any demographic or cohort. For example, the subjects may be of any sex, gender, age, ethnicity, or race. In some cases, the plurality of subjects may include subjects associated with, or belonging to, a population or cohort of interest. By population or cohort of interest is meant a group of people banded together or treated as a group, such as a specific demographic of individuals. For example, the cohort of interest may be individuals who share a particular source of drinking water (such as, e.g., groundwater), or who work in a certain profession (e.g., firefighting). In some instances, the plurality of subjects may consist of only subjects belonging to a cohort of interest.

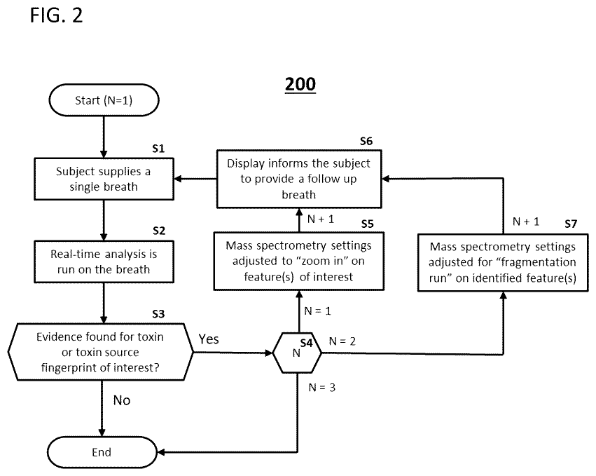

FIG. 2 provides a depiction of a method for dynamically adjusting breath collection automatically based on real-time feedback in accordance with an embodiment of the invention. At step S1, the subject supplies one or more initial breaths to the mass spectrometry analyzer for analysis. At step S2, real-time analysis is performed on the measurements generated by the mass spectrometry analyzer in order to identify one or more toxins or toxic compound or source fingerprints of interest. In some cases, automatic polarity switching and/or ionization agent (e.g., ionizing fluid) switching may be occurring periodically during step S2 in order to generate a more comprehensive and detailed breath fingerprint, enabling a wider range of toxins or toxic compound or source fingerprints of interest to be detected/identified within the breath sample. At step S3, a check is done as to whether there is evidence for the presence of a toxic compound (i.e., toxin) or fingerprint of interest. For compounds of interest, any relevant m/z signal above a predetermined level associated with noise may be considered evidence of the compound of interest. For fingerprints of interest generated using a machine learning model, if a minor adjustment of a small number of m/z signals (e.g., the intensity and/or m/z value of the signals) would result in the identification of a fingerprint of interest, there may be considered evidence of the fingerprint of interest. In some instances, the toxic compound or toxin source fingerprints of interest (generated using machine learning techniques, as discussed in greater detail below) may be fed into a machine learning model that is configured/trained to perform one or more of the real-time automatic dynamic adjustment methods as discussed above (i.e., SIM, fragmentation, ionization agent switching, and/or polarity switching) in order to verify or distinguish between toxic compound or toxin source fingerprints for which evidence is found thereof. If evidence is found for a compound or fingerprint of interest, and SIM has not yet been performed (step S4), the mass spectrometry analyzer is automatically adjusted (using, e.g., the dynamic adjustment machine learning model) to “zoom in” (e.g., limit detection to) one or more features of interest at step S5. The features of interest may be determined using the dynamic adjustment machine learning model. For example, compounds for which a minor alteration in detected intensity would change the identified fingerprint of interest may be classified as features of interest and “zoomed in” on. At step S6, a visual display, such as a liquid crystal display (LCD) screen, prompts the subject to provide one or more additional breaths to the mass spectrometry analyzer. Steps S1 and S2 are then repeated, and the subject provides another breath or set of breaths for which real-time analysis is performed. At step S3, a check is done as to whether there is still evidence for the presence of a toxic compound or toxic compound or source fingerprint after the measurements for the “zoomed in” on compound or compounds are received or updated. If evidence for the presence of a toxic compound or toxic compound or source fingerprint of interest is still present after SIM, the mass spectrometry analyzer is automatically configured to perform fragmentation for one or more features of interest at step S7. Steps S6, S1, and S2 are then repeated in order to verify the presence of the compound or fingerprint of interest, and the assay is ended. In some cases, the subject may be prompted to provide an additional breath or set of breaths prior to SIM, during SIM, and/or during fragmentation measurements as needed (e.g., at step S4). For example, if a trained machine learning algorithm or an operator monitoring breath collection determines an individual breath or set of breaths is not of a sufficient quality, another breath or set of breaths may be provided without resetting the automatic dynamic breath collection process. In some cases, the subject may be prompted to provide multiple breaths or series of breaths to support SIM (e.g., to enhance the statistical significance of results) or to gather additional data for deep learning, as described in greater detail below. Additionally, steps can be performed in which automatic dynamic ionization agent switching and/or polarity switching is used to verify the presence of the compound or fingerprint of interest.

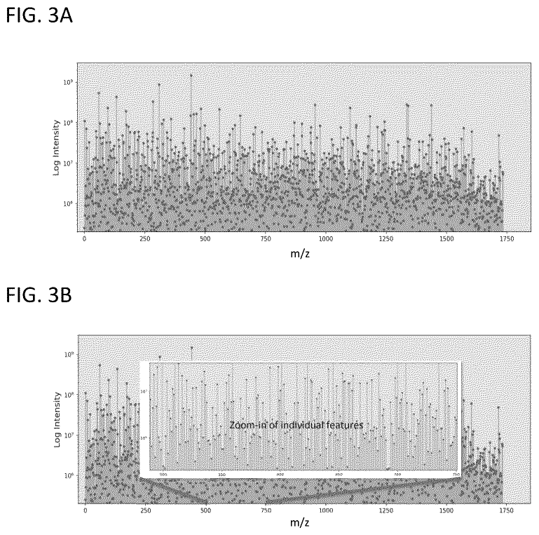

FIGS. 3A and 3B provide an example of SIM. In FIG. 3A, a range from 0 m/z to roughly 1750 m/z is measured in a single scan. In FIG. 3B, a smaller range from roughly 500 m/z to 750 m/z is measured in a single scan, allowing for greater sensitivity and the distinction of compounds similar in m/z value.

### Data Generation and Analysis

As described above, embodiments of the methods include analyzing breath samples from a plurality of subjects with a secondary electrospray ionization-high-resolution mass spectrometry analyzer to generate breath assay data associated with each subject. In some embodiments, the breath assay data may be processed in order to generate a plurality of breath biopsy output files, as described below. The methods and techniques by which a breath biopsy file may be generated and analyzed, in accordance with embodiments of the invention, may vary. In embodiments where the breath assay includes mass spectrometry (i.e., where the breath analyzer includes a mass spectrometry analyzer), breath assay data may be generated and analyzed in real-time, e.g., as described in U.S. Provisional Application Ser. Nos. 63/359,134 and 63/416,185 (Attorney docket nos. DIAG-003PRV and DIAG-003PRV2, respectively) as well as PCT application serial No. PCT/US2023/027001 (Attorney docket no. DIAG-003WO); the disclosures of which are herein incorporated by reference.

As summarized above, methods of assaying a breath sample from a subject for the presence of one or more toxic compounds (e.g., TCE or metabolites thereof) are provided. In embodiments where the breath assay includes mass spectrometry such as, e.g., SESI-MS, the breath sample may be assayed by a mass spectrometry analyzer to generate a breath biopsy output file. In some embodiments, the breath biopsy output file is a RAW file. By RAW file is meant a file that has not been compressed, encrypted, or processed. The breath biopsy output file (e.g., RAW file) may then be automatically detected. The automatically detected breath biopsy output file may then be associated with an identifier of the subject to produce an identifier associated breath biopsy output file. In some embodiments of the methods, associating the automatically detected generated breath biopsy output file with an identifier of the subject includes: receiving an identifier from the subject; and confirming that the generated breath biopsy output file is from analysis of the breath sample obtained from the subject. In some embodiments, computer code (e.g., a program) may be configured to automatically detect the breath biopsy output file and, e.g., prompt a technician or operator regarding the association of the generated output file with a subject (e.g., an identifier of the subject, such as an identification number or code). The technician or operator may then confirm the detected breath biopsy output file is from analysis of the breath sample obtained from the subject and the automatically detected breath biopsy output file may then be associated with the identifier of the subject to produce an identifier associated breath biopsy output file. In some instances, the identifier is associated with the automatically detected generated breath biopsy output file by a human operator, while in other instances the identifier is associated with the automatically detected generated breath biopsy output file by a program (e.g., after confirmation). In other cases, the automatically detected breath biopsy output file is automatically associated with the subject identifier without confirmation from a human operator or technician (e.g., by a program).

As discussed above, a breath biopsy output file (e.g., RAW file) may be automatically detected and subsequently associated with the subject (i.e., an identifier of the subject) to produce an identifier associated breath biopsy output file. The identifier of the subject may vary, where examples of identifiers include, but are not limited to alpha/numeric identifiers (e.g., an identification number or a string of letters and/or numbers), codes such as, e.g., QR codes, barcodes, etc. In some embodiments, the identifier may identify the subject through association with identifying information of the subject such as, but not limited to, the subject's full legal name, contact information, home address, social security number, etc. In these embodiments, the association may occur in a database or in a datasheet (e.g., wherein the identifying information may be found by searching for the identifier). In these cases, it may be relatively difficult or impossible to associate the identifying information of the subject with the identifier without access to the database or the datasheet (i.e., the database or datasheet is secured and/or protected). In some embodiments, the identifier is generated for or assigned to the subject during the session or appointment in which the breath sample is collected (and, e.g., subsequently analyzed wherein the breath biopsy output file is produced). In other embodiments, the identifier is generated for or assigned to the subject before the session or appointment in which the breath sample is collected. In these embodiments, the subject may provide their identifying information through any number of means including, e.g., by navigating to a web address or via email, wherein an identifier is generated for or assigned to the subject after the subject has provided their identifying information. In these instances, the subject may provide the identifier to a technician or operator prior to the collection and analysis (i.e., assaying) of the breath sample of the subject. For example, the subject may provide a QR code to an operator or technician, wherein by scanning the QR code the identifier is received from the subject. In some instances, the identifier may be automatically generated for or assigned to the subject after the subject has provided their identifying information. In some embodiments, the subject may fill in or submit an initial health information questionnaire that may be associated with the identifier of the subject. In some embodiments, the method includes associating the identifier with a prior health record of the subject.

After the identifier associated breath biopsy output file is produced (e.g., as described above), the file may be converted to an open XML-based format such as, e.g., mzML format. In some embodiments, metadata associated with the identifier associated breath biopsy output file may be obtained. The obtained metadata may include, but is not limited to, the subject's identifier and/or identifying information, a health questionnaire submitted by the subject, mass spectrometer status/settings, temperature, humidity, etc. In some embodiments, the metadata is saved in a file (e.g., a logfile) associated with the identifier associated breath biopsy output file (e.g., labeled with the subject's identifier, a timestamp, a lab identifier, a machine identifier, etc.). In some embodiments, a technician may be enabled to enter comments to the metadata file if desired (e.g., indicating the breath sample assayed was contaminated). The metadata file may be in a readable format such as, e.g., JSON, XML, CSV, CSON, TXT, etc. In some embodiments, an intuitive data set is generated from the identifier associated (and, e.g., converted) breath biopsy output file. The intuitive data set may be structured and formatted in order to be compatible with the subsequent steps of the invention. For example, the intuitive data set may be structured and formatted in order to train a machine learning model, as discussed in greater detail below. In some instances, the intuitive data set (e.g., and the metadata file associated therewith) is used to generate a health evaluation as described in greater detail below. In some embodiments, the intuitive data set is generated, at least in part, by reducing the data of the identifier associated breath biopsy output file.

In embodiments where generating the intuitive data set includes reducing the data of the identifier associated (and, e.g., converted) breath biopsy output file, the reduction may vary. In some embodiments, the reduction may depend on one or more components of the training and/or configuration of the machine learning model, as discussed in greater detail below. In embodiments wherein the breath sample is collected directly from the subject (i.e., without a phase transition), the reduction may include the processing step of automatically identifying individual breaths in the sample. In some embodiments, a breath duration is determined for each identified breath indicating the time from the onset of the breath to the end of the breath.

In some embodiments, the reduction process may include the step of automatically identifying all features (i.e., peaks or measurements) of the breath sample from the identifier associated breath biopsy output file. Statistical measures of the identified features may then be determined. The automatically identified features of the breath sample may be matched or associated with compounds, e.g., using the mass to charge ratio (m/z) of each peak and/or the time from the beginning of an identified breath each peak was generated. In some embodiments, a value of abundance is generated for the identified peaks matched or associated with compounds, e.g., using the intensity of each peak and/or the identity of the associated compound. By value of abundance is meant a quantitative value related to, in some instances indicating, the number or amount of the matched or associated compound in the breath sample.

In some embodiments, the reduction may include the step of omitting or excluding (e.g., deleting) data determined to not be necessary for further analysis (e.g., the training of a machine learning model, as described below) after a processing step or processing steps (e.g., as described above) have been performed or executed. For example, after the processing step of automatically identifying breaths in the sample data has been executed, data (e.g., peaks or scans) not generated during an identified breath may be deleted or omitted.

In some embodiments, an overview of the results of the breath sample assay may be generated from the data of the converted identifier associated breath biopsy output file or the intuitive data set generated therefrom. For example, the overview may include the number of peaks found, the peaks found at different m/z values over the time the assay was run, total ion current, various statistical analyses, the number of matched or associated compounds detected per identified breath, an intensity distribution, a histogram of the number of features per m/z value, etc. In some instances, the overview may additionally contain data from the breath collection device or system. For example, the overview may contain the flow rate a breath sample was collected at, the volume of a breath sample, the temperature of a breath sample, a value of abundance of water vapor or carbon dioxide in a breath sample (e.g., the percentage of water vapor or carbon dioxide in a breath sample), etc. In some embodiments, the overview may display or convey the results of the breath sample assay on a per assayed breath basis. In some cases, this may allow outlier breaths to be identified and potentially excluded from the health evaluation in order to, e.g., enhance the accuracy of the results. In some cases, outlier breaths are identified using a machine learning model such as, e.g., a machine learning model trained or including architecture as described below. In some instances, outlier breaths may be identified using a rules-based system. In some embodiments, the overview may indicate potential problems including, but not limited to, problems associated with the breath sample quality, possible contamination, etc. In these instances, an operator or technician may choose to adjust the machine configuration or capture additional breath samples based, at least in part, on feedback provided by the overview. In some cases, the overview may be generated in real time. By real time is meant that the overview is generated during or immediately following the breath sample assay (e.g., during collection of the breath sample or while the breath sample is being analyzed using, e.g., a mass spectrometry analyzer). In some instances, the overview is generated in two hours or less. In some cases, the overview is generated in one hour or less, such as thirty minutes or less, or twenty minutes or less, or ten minutes or less, or five minutes or less, or one minute or less. In some instances, one or more of the identifier associated breath biopsy output file, the intuitive data set generated from the breath biopsy output file, the metadata file associated with the breath biopsy output file, or the overview of the results of the breath sample assay may be saved or archived to a database such as, e.g., a database including a data warehouse. In some cases, one or more non-breath assay health records of the subject are associated with the identifier of the identifier associated breath biopsy output file, the intuitive data set, the metadata file, and/or the overview. The one or more non-breath assay health records of the subject are then saved or archived to the database (e.g., data warehouse) with the breath biopsy files.

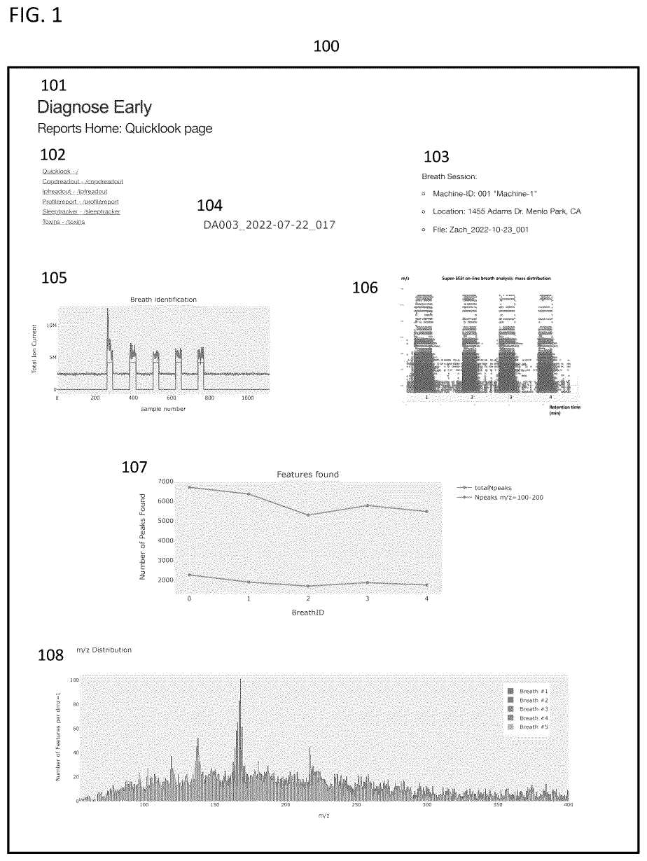

FIG. 1 provides a depiction of an overview of the results obtained from an identifier associated breath biopsy output file in accordance with an embodiment of the invention. Overview (i.e., Quicklook) 100 includes header 101 and selectable menu 102 provided to assist a viewer in navigating between sections of a health evaluation when, e.g., the evaluation and the overview are both displayed on an electronic viewing device (e.g., a computer or a smart phone). Session summary 103 provides information pertaining to the session in which the breath sample assay was performed. The overview further includes the identifier of the subject 104 as well as various charts and graphs depicting data of the intuitive data set generated from the breath sample assay. Graph 105 depicts the TIC per sample number (i.e., scan number), with the orange line indicating sample numbers wherein an exhaled breath is received by the mass spectrometer. Graph 106 depicts the m/z value of compounds detected by the mass spectrometer over time. Graph 107 depicts the total number of peaks found per identified exhaled breath received by the mass spectrometer. Graph 108 depicts a histogram of the number of features detected per m/z value, with colors indicating which identified exhaled breath each bin belongs to. In some cases, the overview may be generated, at least in part, using a trained machine learning algorithm. In these cases, the overview may further indicate breaths determined to not be of a sufficient quality that were excluded from downstream analysis (e.g., to generate a health evaluation).

### Generating a Toxin Exposure Assessment

The toxin exposure assessment is a qualitative or quantitative determination regarding whether one or more toxins or toxin associated compounds (e.g., metabolites of the one or more toxins) or toxic compound or source fingerprints are present in a breath sample obtained from a subject, where the resultant determination is employed as an indicator of whether the subject from which the assayed breath sample was obtained has been exposed to the one or more toxins (or, e.g., has a biological condition induced by said exposure). By toxin is meant an agent (e.g., a compound) known or suspected of being harmful to the subject being assayed. Toxins and toxin associated compounds that may be detected in a breath sample, in accordance with embodiments of the methods, may vary and include but are not limited to those found below. In some instances, the one or more toxins includes one or more carcinogens. Carcinogens of interest include, but are not limited to, carcinogens classified as being Group 1 carcinogens by the International Agency for Research on Cancer (IARC). A Group 1 classification indicates that an agent (e.g., a compound) exhibits sufficient evidence of carcinogenicity in humans. Examples of Group 1 carcinogens include those listed in Table 1, below:

TABLE 1
Group 1 carcinogens

| | | Molecular | Monoisotopic | |
|---|---|---|---|---|
| Compound name | CAS | formula | mass | [M + H]+ |
| 4,4′-Methylenebis(2-chloroaniline) | 101-14-4 | C13H12Cl2N2 | 266.0377538 | 267.0450303 |
| (MOCA) | | | | |
| Tamoxifen | 10540-29-1 | C26H29NO | 371.2249145 | 372.232191 |
| 1,3-Butadiene | 106-99-0 | C4H6 | 54.04695019 | 55.05422666 |
| Polychlorinated biphenyls | 1336-36-3 | C12H4Cl6 | 357.844416 | 358.8516925 |
| Semustine [1-(2-Chloroethyl)-3-(4- | 13909-09-6 | C10H18ClN3O2 | 247.1087545 | 248.116031 |
| methylcyclohexyl)-1-nitrosourea, | | | | |
| Methyl-CCNU] | | | | |
| Melphalan | 148-82-3 | C13H18Cl2N2O2 | 304.0745332 | 305.0818097 |
| 2,3,7,8-Tetrachlorodibenzo-para- | 1746-01-6 | C12H4Cl4O2 | 319.89654 | 320.9038165 |
| dioxin | | | | |
| Methoxsalen (8-methoxypsoralen) | 298-81-7 | C12H8O4 | 216.0422587 | 217.0495352 |
| plus ultraviolet A radiation | | | | |
| Treosulfan | 299-75-2 | C6H14O8S2 | 278.0130098 | 279.0202862 |
| Chlorambucil | 305-03-3 | C14H19Cl2NO2 | 303.0792842 | 304.0865607 |
| Aristolochic acid | 313-67-7 | C17H11NO7 | 341.0535517 | 342.0608282 |
| Etoposide | 33419-42-0 | C29H32O13 | 588.1842911 | 589.1915675 |
| Azathioprine | 446-86-6 | C9H7N7O2S | 277.0381937 | 278.0454701 |
| Chlornaphazine | 494-03-1 | C14H15Cl2N | 267.0581549 | 268.0654314 |
| Cyclophosphamide | 50-18-0 | C7H15Cl2N2O2P | 260.0248201 | 261.0320966 |
| Benzo[a]pyrene | 50-32-8 | C20H12 | 252.0939004 | 253.1011768 |
| Thiotepa | 52-24-4 | C6H12N3PS | 189.0489556 | 190.056232 |
| Bis(chloromethyl)ether | 542-88-1 | C2H4Cl2O | 113.9639201 | 114.9711966 |
| Busulfan | 55-98-1 | C6H14O6S2 | 246.0231805 | 247.030457 |
| Diethylstilbestrol | 56-53-1 | C18H20O2 | 268.1463299 | 269.1536063 |
| 2,3,4,7,8-Pentachlorodibenzofuran | 57117-31-4 | C12H3Cl5O | 337.862653 | 338.8699295 |
| 3,4,5,3′,4′-Pentachlorobiphenyl | 57465-28-8 | C12H5Cl5 | 323.883389 | 324.8906655 |
| (PCB-126) | | | | |
| Hexachlorocyclohexane | 58-89-9 | C6H6Cl6 | 287.860066 | 288.8673425 |
| Phenacetin | 62-44-2 | C10H13NO2 | 179.0946287 | 180.1019051 |
| Benzene | 71-43-2 | C6H6 | 78.04695019 | 79.05422666 |
| Vinyl chloride | 75-01-4 | C2H3Cl | 61.9923278 | 62.99960427 |
| Ethylene oxide | 75-21-8 | C2H4O | 44.02621475 | 45.03349121 |
| 1,2-Dichloropropane | 78-87-5 | C3H6Cl2 | 111.9846556 | 112.9919321 |
| Trichloroethylene | 79-01-6 | C2HCl3 | 129.914383 | 130.9216595 |
| Pentachlorophenol | 87-86-5 | C6HCl5O | 263.847003 | 264.8542795 |
| 2-Naphthylamine | 91-59-8 | C10H9N | 143.0734993 | 144.0807758 |
| 4-Aminobiphenyl | 92-67-1 | C12H11N | 169.0891494 | 170.0964258 |
| Benzidine | 92-87-5 | C12H12N2 | 184.1000484 | 185.1073249 |
| ortho-Toluidine | 95-53-4 | C7H9N | 107.0734993 | 108.0807758 |

In the above Table 1 the Chemical Abstracts Service (CAS) Registry Number, molecular formula, monoisotopic mass, and the [M+H]+ ion mass are provided for each listed Group 1 carcinogen.

Carcinogens of interest also include, but are not limited to, carcinogens classified as Group 2A carcinogens by the IARC. Group 2A classification indicates that an agent (e.g., a toxin) is probably carcinogenic. Examples of Group 2A carcinogens include those listed in Table 2, below.

TABLE 2
Group 2A carcinogens

| | | Molecular | Monoisotopic | |
|---|---|---|---|---|
| Compound name | CAS | formula | mass | [M + H]+ |
| Styrene | 100-42-5 | C8H8 | 104.0626003 | 105.06988 |
| Epichlorohydrin | 106-89-8 | C3H5ClO | 92.0028925 | 93.010169 |
| Glycidyl methacrylate | 106-91-2 | C7H10O3 | 142.0629942 | 143.07027 |
| Ethylene dibromide | 106-93-4 | C2H4Br2 | 185.86798 | 186.87526 |
| Acrolein | 107-02-8 | C3H4O | 56.02621475 | 57.033491 |
| Glyphosate | 1071-83-6 | C3H8NO5P | 169.0140094 | 170.02129 |
| Pioglitazone | 111025-46-8 | C19H20N2O3S | 356.1194637 | 357.12674 |
| 1,3-Propane sultone | 1120-71-4 | C3H6O3S | 122.0037652 | 123.01104 |
| Tetrafluoroethylene | 116-14-3 | C2F4 | 99.99361265 | 101.00089 |
| Malathion | 121-75-5 | C10H19O6PS2 | 330.0360677 | 331.04334 |
| Tris(2,3-dibromopropyl) | 126-72-7 | C9H15Br6O4P | 691.58082 | 692.5881 |
| phosphate | | | | |
| Tetrachloroethylene | 127-18-4 | C2Cl4 | 163.875411 | 164.88269 |
| 1-(2-Chloroethyl)-3-cyclohexyl-1- | 13010-47-4 | C9H16ClN3O2 | 233.0931045 | 234.10038 |
| nitrosourea (CCNU) | | | | |
| ortho-Anisidine | 90-04-0 | C7H9NO | 123.0684139 | 124.07569 |
| 3,3′,4,4′-Tetrachloroazobenzene | 14047-09-7 | C12H6Cl4N2 | 317.928509 | 318.93579 |
| Aniline | 142-04-1 | C6H7N | 93.05784923 | 94.065126 |
| 2-Mercaptobenzothiazole | 149-30-4 | C7H5NS2 | 166.9863415 | 167.99362 |
| Bischloroethyl nitrosourea | 154-93-8 | C5H9Cl2N3O2 | 213.0071819 | 214.01446 |
| Dibenzo[a,l]pyrene | 191-30-0 | C24H14 | 302.1095504 | 303.11683 |
| Indium phosphide | 22398-80-7 | InP | 145.8776408 | 146.88492 |
| Dibenz[a,j]acridine | 224-42-0 | C21H13N | 279.1047994 | 280.11208 |
| Adriamycin | 23214-92-8 | C27H29NO11 | 543.1740607 | 544.18134 |
| Captafol | 2425-06-1 | C10H9Cl4NO2S | 346.91081 | 347.91809 |
| Cyclopenta[cd]pyrene | 27208-37-3 | C18H10 | 226.0782503 | 227.08553 |
| Teniposide | 29767-20-2 | C32H32O13S | 656.1563622 | 657.16364 |
| Chloral hydrate | 302-17-0 | C2H3Cl3O2 | 163.919862 | 164.92714 |
| Azacitidine | 320-67-2 | C8H12N4O5 | 244.0807695 | 245.08805 |
| Diazinon | 333-41-5 | C12H21N2O3PS | 304.1010507 | 305.10833 |
| Procarbazine | 671-16-9 | C12H19N3O | 221.1528122 | 222.16009 |
| 5-Methoxypsoralen | 484-20-8 | C12H8O4 | 216.0422587 | 217.04954 |
| DDT (4,4′- | 50-29-3 | C14H9Cl5 | 351.914689 | 352.92197 |
| dichlorodiphenyltrichloroethane) | | | | |
| Urethane | 51-79-6 | C3H7NO2 | 89.04767847 | 90.054955 |
| Dibenz[a,h]anthracene | 53-70-3 | C22H14 | 278.1095504 | 279.11683 |
| 1,2-Dimethylhydrazine | 540-73-8 | C2H8N2 | 60.06874826 | 61.076025 |
| Chlorozotocin | 54749-90-5 | C9H16ClN3O7 | 313.0676776 | 314.07495 |
| N-Nitrosodiethylamine | 55-18-5 | C4H10N2O | 102.0793129 | 103.08659 |
| 1-Nitropyrene | 5522-43-0 | C16H9NO2 | 247.0633285 | 248.07061 |
| Glycidol | 556-52-5 | C3H6O2 | 74.03677943 | 75.044056 |
| Chloramphenicol | 56-75-7 | C11H12Cl2N2O5 | 322.0123269 | 323.0196 |
| Vinyl bromide | 593-60-2 | C2H3Br | 105.94181 | 106.94909 |
| 2,3,3′,4,4′,5′-Hexabromobiphenyl | 59536-65-1 | C12H4Br6 | 621.54133 | 622.54861 |
| N-Nitrosodimethylamine | 62-75-9 | C2H6N2O | 74.04801282 | 75.055289 |
| Diethyl sulfate | 64-67-5 | C4H10O4S | 154.02998 | 155.03726 |
| Methyl methanesulfonate | 66-27-3 | C2H6O3S | 110.0037652 | 111.01104 |
| N,N-Dimethylformamide | 68-12-2 | C3H7NO | 73.05276385 | 74.06004 |
| N-Methyl-N-nitrosourea | 684-93-5 | C2H5N3O2 | 103.0381764 | 104.04545 |
| N-Methyl-N′-nitro-N- | 70-25-7 | C2H5N5O3 | 147.039239 | 148.04652 |
| nitrosoguanidine | | | | |
| 1,1,1-Trichloroethane | 71-55-6 | C2H3Cl3 | 131.930033 | 132.93731 |
| 6-Nitrochrysene | 7496-02-8 | C18H11NO2 | 273.0789786 | 274.08626 |
| Vinyl fluoride | 75-02-5 | C2H3F | 46.02187826 | 47.029155 |
| Dichloromethane | 75-09-2 | CH2Cl2 | 83.9533555 | 84.960632 |
| Chloral | 75-87-6 | C2HCl3O | 145.909298 | 146.91657 |
| N-Ethyl-N-nitrosourea | 759-73-9 | C3H7N3O2 | 117.0538265 | 118.0611 |
| 2-Amino-3-methylimidazo[4,5- | 76180-96-6 | C11H10N4 | 198.0905463 | 199.09782 |
| f]quinoline | | | | |
| Dimethyl sulfate | 77-78-1 | C2H6O4S | 125.9986798 | 127.00596 |
| Acrylamide | 79-06-1 | C3H5NO | 71.03711378 | 72.04439 |
| Dimethylcarbamoyl chloride | 79-44-7 | C3H6ClNO | 107.0137915 | 108.02107 |
| Tetrabromobisphenol A | 79-94-7 | C15H12Br4O2 | 539.75708 | 540.76436 |
| 2-Nitrotoluene | 88-72-2 | C7H7NO2 | 137.0476785 | 138.05495 |
| 2-Nitroanisole | 91-23-6 | C7H7NO3 | 153.0425931 | 154.04987 |
| 4-Chloro-2-methylaniline | 95-69-2 | C7H8ClN | 141.034527 | 142.0418 |
| Styrene-7,8-oxide | 96-09-3 | C8H8O | 120.0575149 | 121.06479 |
| 1,2,3-Trichloropropane | 96-18-4 | C3H5Cl3 | 145.945683 | 146.95296 |

In the above Table 2 the CAS Registry Number, molecular formula, monoisotopic mass, and the [M+H]+ ion mass are provided for each listed Group 2B carcinogen.

In some instances, the one or more toxins includes a toxin and/or metabolite thereof selected from the group consisting of Per- and Polyfluoroalkyl Substances (PFAS), Trichloroethylene, 1,3-Butadiene, Chlornaphazine, Thiotepa, Bis(chloromethyl) ether, Phenacetin, Benzene, Vinyl Chloride, 1,2-Dichloropropane, 2-Napthylamine, 4-Aminobiphenyl, Ortho Toluidine, Dichloromethane, N,N-Dimethylformamide and Styrene-7,8 oxide.

In some instances, the one or more toxins includes one or more PFAS. PFAS that may be detected include, but are not limited to, those disclosed on the CDCs website (see https://www.cdc.gov/niosh/topics/pfas/default.html) and on the linked pages of the CDCs website. In some instances, PFAS that may be detected include, but are not limited to Perfluorobutanoic acid (PFBA), Perfluoropentanoic acid (PFPeA), Perfluorohexanoic acid (PFHxA), Perfluoroheptanoic acid (PFHpA), Perfluorooctanoic acid (PFOA), Perfluorononanoic acid (PFNA), Perfluorodecanoic acid (PFDA), Perfluorobutanesulfonic acid (PFBS), Perfluoropentanesulfonic acid (PFPeS), Perfluorohexanesulfonic acid (PFHxS), Perfluoroheptanesulfonic acid (PFHpS) and Perfluorooctanesulfonic acid (PFOS).

In some instances, the one or more toxins includes Trichloroethylene and/or a metabolite thereof, such as but not limited to: trichloro acetic acid, dichloroacetic acid, trichloroethanol, 1,2, dichlorovinyl glutathione, 1,2-dichlorovinyl cysteine (DCVC) and chlorothioketene.

In some instances, a qualitative or quantitative determination regarding whether one or more toxins is present in the breath sample is employed as an indicator of whether the subject has been exposed to the one or more toxins. In other instances, a qualitative or quantitative determination regarding whether one or more toxin associated compounds is present in the breath sample is employed as an indicator of whether the subject has been exposed to the one or more toxins. By toxin associated compound(s) is meant the compound(s) are different from the one or more toxins but the presence of the compound(s) in the breath sample indicates a potential exposure of the subject to the one or more toxins. In some instances, the toxin associated compounds may be one or more metabolites of the one or more toxins. In some cases, the toxin associated compounds may be compounds typically found with the one or more toxins that also enter the body when an individual (e.g., the subject) has been exposed to the one or more toxins. For example, a determination regarding the presence of one or more non-toxic (e.g., non-carcinogenic) compounds associated with, e.g., environmental tobacco smoke in the breath sample might be employed as an indicator of whether the subject has been exposed to one or more toxins associated with environmental tobacco smoke.

In some instances, the breath sample is assayed for the presence of one or more toxin associated compounds when the one or more toxins are not present in the breath sample at a detectable level (due to, e.g., the pharmacokinetics of the toxin in the subject) and/or when the one or more toxins are not measurable via breath analysis. In some cases, exposure of a subject to a toxic compound or source is determined by detecting a toxic compound or source fingerprint, as discussed above. In these cases, the m/z peaks or measurements of the fingerprint, whether identified or unidentified, may be associated with compounds typically found with a toxic compound or toxin source (i.e., that the subject is also exposed to) or may be associated with the subject's body (i.e., cells of the subject's body) reacting to the toxic compound or toxin source. For example, the m/z peaks of the toxic compound or toxin source fingerprint may be associated with compounds found in the subject's breath as the result of the subject's immune system, endocrine system, or a developing fetus in the subject's womb reacting with one or more toxic compounds. In embodiments where the toxin is TCE, the m/z peaks of the TCE fingerprint may be associated with compounds found in the subject's breath as the result of the subject's immune system, urinary system (e.g., kidneys), or digestive system (e.g., liver) reacting with TCE or a metabolite thereof. In some cases, the reaction mechanism of the one or more toxic compounds and the cells of the subject's body is known or may be identified. In embodiments where the toxic compound or source fingerprint is identified using a machine learning model (e.g., as discussed in greater detail below), the nature or mechanism of the reaction between the one or more toxic compounds and the cells of the subject's body may be difficult to obtain or extract and/or may be unknown to the individuals implementing the model (e.g., the relationships may be too complexed to be understood or interpreted by a human or the relationships may contained in a component of the machine learning model considered to be a “black box”).

As described above, embodiments of the invention may include a toxin exposure assessment that can be used to determine if the subject has been exposed to a toxic compound or toxin source and to monitor changes in toxin exposure over time in the subject. In some cases, the toxin exposure assessment discloses whether one or more toxins or toxin associated compounds (e.g., metabolites of the one or more toxins) are present in a breath sample obtained from a subject. In some embodiments, a threshold or limit of detection is used to determine if a toxin or toxic compound or toxin source fingerprint is present in the breath sample at a relevant (e.g., statistically relevant) concentration or intensity. In some embodiments, the toxin exposure assessment may be generated based on a value of abundance of one or more toxic compounds as determined by the breath sample assay. By value of abundance is meant a quantitative value relating to, in some instances indicating, the number or amount of one or more toxic compounds in the breath sample. In some instances, the value of abundance is expressed in normalized arbitrary units. In some cases, the value of abundance is expressed in units of concentration. For example, the value of abundance may be expressed in parts per million (ppm). The toxin exposure assessment may be generated based at least in part on whether one or more toxic compounds or toxic compound or source fingerprints is detected in the breath sample. In some instances, the toxin exposure assessment is generated based at least in part on whether two or more different toxic compounds or toxic compound or source fingerprints are detected in the breath sample, such as three or more, or six or more, or ten or more, or twenty or more, or fifty or more different toxic compounds or fingerprints. In some embodiments, the toxin exposure assessment may be generated based at least in part on whether TCE and one or more additional TCE metabolites are detected in the breath sample. In some embodiments, the value of abundance is provided as a level or degree of detection. For example, a given toxin or toxin associated compound may be assigned a detection level of, e.g., not detected, trace, modest, strong, etc. as determined by the breath sample assay.

In some embodiments, the assigned detection level as described above is based, at least in part, on the toxicity of the toxin to the subject. By toxicity is meant the ability or potential for the toxin to cause harm to the subject. In some embodiments, the assigned detection level is based, at least in part, on pharmacokinetic properties of the toxin in the subject. Pharmacokinetic properties of interest include, but are not limited to, absorption properties, distribution properties, metabolic properties, and excretion or elimination properties. For example, the assigned detection level may accommodate for the distribution the subject may have in the lungs in comparison to the rest of the subject's body. In another example, the assigned detection level may accommodate for the clearance rate (i.e., elimination rate) of the toxin from the subject. In some embodiments, the toxicity of each toxin and/or pharmacokinetic properties of each toxin are reflected in a qualitative or quantitative component of the toxin exposure assessment separate from the value of abundance (e.g., separate from the assigned detection level).

In some instances, the toxin exposure assessment includes a determination about the level of one or more toxins (i.e., directly measured or measured using a fingerprint as discussed above) in a breath sample relative to a baseline. The baseline may vary, and in some instances includes a cohort average value, such as an average level or amount of a given toxin found in a population or cohort of interest. By population or cohort is meant a group of people banded together or treated as a group, such as the categories of professionals listed above, e.g., fire fighters, a group of people living in a specified locality, a group of people in the same age range, etc. In some instances, the baseline includes a prior value obtained from the subject, e.g., a value obtained from the subject 1 day prior to the assay, 1 week prior to the assay, 1 month prior to the assay, 6 months prior to the assay, 1 year prior to the assay, 5 years prior to the assay, etc. In such instances, the toxin exposure assessment may indicate a temporal change of the level of a toxin found in the breath of the subject, i.e., how the level of a toxin or multiple toxins has changed over time for the subject.

In some instances, the toxin exposure assessment includes a determination about the level of exposure of the subject supplying the breath sample to one or more toxic compounds or toxin sources. In some embodiments, the exposure determination is generated by assaying breath samples provided by one or more individuals known to have been exposed to a toxic compound or toxin source to produce breath biopsy data (i.e., breath assay data). In these instances, the time of the exposure, the duration of the exposure and/or one or more other conditions affecting the extent or level of exposure of the one or more individuals to the toxic compound or toxin source is known. The breath assay data of the one or more individuals with known exposure events may then be used to generate a breath fingerprint of exposed individuals. The exposed breath fingerprint may include measurements of a toxin (or, e.g., a metabolite of a toxin) and/or one or more measurements of other compounds that are not toxins or known metabolites thereof (including, e.g., unidentified compounds, volatile organic compounds [VOCs], etc.) as described above. In some embodiments, the exposed breath fingerprint is generated using machine learning techniques, e.g., as described in greater detail below. The generated exposed breath fingerprint may then be used to determine the level of exposure of a subject of unknown exposure status using a breath sample supplied by the subject. The determined level of exposure may include the time of exposure of the subject, the duration of exposure and/or any other conditions of a potential exposure of the subject based on the known exposure circumstances of the one or more individuals from which the exposed breath fingerprint was generated. In some cases, the one or more individuals with known exposure events may be assayed at multiple timepoints in order to generate exposed breath fingerprints specific to the amount of time that has passed since an exposure event. The multiple elapsed time specific fingerprints may then be used to determine the amount of time that has passed since a subject of unknown exposure status has been exposed to a toxic compound or toxin source.

As described above, the toxin exposure assessment may include a determination about the level of exposure of the subject supplying the breath sample to one or more toxic compounds or toxin sources. In some cases, the determined level of exposure is based, at least in part, on the toxicity of the toxin to the subject or pharmacokinetic properties of the toxin in the subject as described. In some embodiments, the determined level of exposure is based, at least in part, on data obtained from a source of toxins. A source of toxins may include, but is not limited to, a municipal water supply, a consumer product (e.g., carpets, food packaging, furnishings, cosmetics, outdoor gear, clothing, adhesives and sealants, non-stick cookware, etc.), a location (e.g., a place of work or a location where the subject may perform a task associated with employment), or an event (e.g., a house fire, a forest fire, an industrial fire, etc.) where toxins are present. The source may include one or more toxins with different abundances (e.g., concentrations) relative to one another. The specific toxins and the relative abundance of each toxin in a specific source of toxins may include the fingerprint of the source of toxins. In some cases, the fingerprint of the source of toxins is identified in the subject's breath using a machine learning model, as discussed in greater detail below. In some embodiments, the fingerprint of a source of toxins is used to generate the exposure determination. For example, a source of toxins may have a fingerprint characterized by the concentration of a first toxin that is twice as high as the concentration of a second toxin and where a third toxin is absent. In this case, the exposure determination may be made, e.g., by comparing the different toxins (i.e., measured directly or indirectly via a toxin fingerprint) in a breath sample to different toxins in a source of toxins. In some embodiments, the exposure determination may indicate the likelihood that the subject has been exposed to the source of toxins.

The subject may be assayed at two or more timepoints to generate two or more toxin exposure assessments. In some instances, the subject may be assayed at three or more timepoints to generate three or more toxin exposure assessments, such as four or more, or five or more, or ten or more. The two or more timepoints may be at least a day apart from each other, such as at least a week apart from each other, or at least a month apart from each other, or at least a year apart from each other. In some instances, a first timepoint of the two or more timepoints may occur after a potential exposure of the subject to a source of toxins. In other cases, a first timepoint of the two or more timepoints occurs before a potential exposure of the subject to a source of toxins in order to, e.g., function as a baseline as discussed above. In these instances, the first timepoint may occur prior to the subject initiating employment (e.g., as a firefighter) or moving to a new location. The subject may be assayed (i.e., a timepoint may occur) every set number of days or months while they are at a certain location or working a certain profession (e.g., firefighting). In instances where the subject is assayed at two or more timepoints to generate two or more toxin exposure assessments the two or more toxin exposure assessment may be used to, e.g., determine changes in exposure of the subject to one or more toxins over time, or determine a clearance time of one or more toxins from the subject.

In some embodiments, the toxin exposure assessment may include a metabolic profile or metabolic profiles of the breath sample of the subject. By metabolic profile is meant a higher-level view of the state of metabolic pathways or presence of various groupings of compounds (e.g., toxins) in the individual at the time the breath is collected. A metabolic profile may compare a particular breath or breaths obtained from the subject to a baseline (e.g., as described above). Abnormal metabolic profiles may help identify toxin exposures, the causes of certain symptoms, screen for disease, and guide treatment regimens. The metabolic profiles may be tailored to assist medical professionals with decision making. For example, compounds associated with specific toxin sources (e.g., toxin source signatures), specific diseases or symptoms, or falling under the same category of toxin, may be grouped together and intuitively displayed, e.g., with their determined levels or values of abundance.

In some instances, the toxin exposure assessment is generated using non-breath assay data. The non-breath assay data may include data obtained from the subject, as discussed below. In some embodiments, the non-breath assay data may affect any of the determinations and findings of the toxin exposure assessment as discussed above. For example, physiological data obtained from a wearable device may be used determine the amount of time that has passed since an exposure event and/or be used to draw specific conclusions about the clearance rate (i.e., elimination rate) of toxins from the subject. As discussed above, a toxin exposure assessment may be generated for a subject from breath assay data or non-breath assay data. In some instances, the toxin exposure assessment is generated in real time. By “real time” is meant the toxin exposure assessment is generated during or immediately following the breath sample assay (e.g., during collection of the breath sample or while the breath sample is being assayed using, e.g., a mass spectrometer). In some instances, the toxin exposure assessment is generated in two hours or less. In some cases, the toxin exposure assessment is generated in one hour or less, such as thirty minutes or less, or twenty minutes or less, or ten minutes or less, or five minutes or less, or one minute or less following the breath sample assay. In some instances, the toxin exposure assessment is generated in real-time using a brief biopsy output file, e.g., as described in United States Provisional Application Serial Nos. 63/359,134 and 63/416,185 (Attorney docket nos. DIAG-003PRV and DIAG-003PRV2, respectively); the disclosures of which are herein incorporated by reference.

The toxin exposure assessment may include one or more personalized insights. A personalized insight may vary and includes, but is not limited to, the detection of an anomaly, a classification, the detection of a cluster, etc. In some instances, the personalized insight includes an insight regarding the subject individually. In other cases, the personalized insight includes an insight regarding a group or cohort in which the subject belongs. In embodiments where the insight includes the detection of an anomaly, the insight may include the identification of unusual data. For example, the insight may be that a specific toxin is detected at a higher level or concentration than usual (e.g., when compared to a baseline as described above). In embodiments where the insight includes a classification, the insight may include the identification of a group with similar data to the subject and, e.g., assigning and comparing the results and data of the subject to the group. For example, the insight may be that the subject has less exposure to a toxin than 80% of a defined cohort for that subject, (e.g., firefighters when the subject is a firefighter). In embodiments where the insight includes the detection of a cluster, the insight may include finding groups with similar results. For example, the insight may be that a city or neighborhood has the highest rate of exposure to a toxin.

In some embodiments, the toxin exposure assessment may include notes or explanations aiding the subject, or a person associated with the subject, in interpreting the results of the toxin exposure assessment. For example, the assessment may include an explanation regarding typical manners in which an individual may be exposed to a toxin (e.g., sources of the toxin), steps the subject may take to avoid potential future exposure to the toxin, and/or diseases and conditions associated with exposure of an individual to the toxin. In some cases, the toxin exposure assessment may include notes indicating information relevant specifically to the subject. For example, the assessment may include a note indicating a difference between the level of one or more toxins in the breath sample relative to a baseline, or symptoms associated with the determined level of one or more toxins in the breath sample (e.g., dizziness, headaches, skin lesions, etc.). In some embodiments, the toxin exposure assessment may include a background section such as, e.g., a background section explaining the purpose of the toxin exposure assessment. In some embodiments, the toxin exposure assessment may include visual means aiding the subject, or a person associated with the subject, in interpreting the findings of the toxin exposure assessment and/or the results of the breath sample assay (e.g., figures, charts, images, etc.).

In some instances, the toxin exposure assessment is generated or obtained using machine learning techniques. In embodiments wherein machine learning techniques are used (e.g., machine learning algorithms and models as described in greater detail below), any of the components the toxin exposure assessment is comprised of such as, e.g., any of the components described above may be generated or obtained using the machine learning techniques. For example, a machine learning model may be trained in order to identify toxic compound or toxin source fingerprints. In some cases, a machine learning model may be trained in order to generate the personalized insight(s) as described above (i.e., the model may detect an anomaly, make the classification, detect of a cluster, etc.).

In some instances, the toxin exposure assessment (e.g., obtained or generated as discussed above) is associated with an identifier of the subject. The identifier of the subject may vary, where examples of identifiers include, but are not limited to alpha/numeric identifiers (e.g., an identification number or a string of letters and/or numbers), codes such as, e.g., QR codes, barcodes, etc. In some embodiments, the identifier may identify the subject through association with identifying information of the subject such as, but not limited to, the subject's full legal name, contact information, home address, social security number, etc. In these embodiments, the association may occur in a database or in a datasheet (e.g., wherein the identifying information may be found by searching for the identifier). In these cases, it may be relatively difficult or impossible to associate the identifying information of the subject with the identifier without access to the database or the datasheet (i.e., the database or datasheet is secured and/or protected). In some instances, the toxin exposure assessment and associated identifier may be saved via local storage and/or cloud storage and, e.g., may be saved to a database such as a data warehouse.

In some embodiments, the toxin exposure assessment may be generated based on correlations and relationships determined from previously saved toxin exposure assessments such as, e.g., the toxin exposure assessments saved to a data warehouse as discussed above. The previously saved toxin exposure assessments may include toxin exposure assessments generated from the subject presently obtaining the toxin exposure assessment and/or toxin exposure assessments generated from other subjects for which one or more toxin exposure assessments were previously obtained. For example, a data warehouse containing previously generated toxin exposure assessments may be used to determine a relationship between toxin exposure and breath assay data and/or non-breath assay data, e.g., a relationship between toxin exposure and health outcomes, the likelihood the subject has been exposed to a specific source of toxins, a pharmacokinetic property of a toxin in the body of a subject, etc. The determined relationship may then be used to generate a subsequent toxin exposure assessment. In some embodiments, the relationship or correlation may be determined, at least in part, using a machine learning algorithm (e.g., by training a machine learning model, as discussed in greater detail below).

In some embodiments, the method further includes suggesting preventative measures based on the toxin exposure assessment, such as, e.g., recommended personal protective equipment (PPE) to avoid potential future exposure to a toxin or a toxin. In some embodiments, the method further includes providing a therapy recommendation to the subject based on the toxin exposure assessment. While the therapy recommendation may vary, in some instances the therapy recommendation includes a detoxication regimen. In some instances, the method further includes administering the therapy to the subject.

The toxin exposure assessment may include information pertaining to the session in which the breath sample assay was performed. For example, the toxin exposure assessment may include, e.g., the date in which the breath sample assay was obtained, an identification number associated with a machine used to assay the breath sample, the location in which the breath sample assay took place (e.g., the address of a pharmacy, clinic, hospital, or primary care physicians office), the number of breaths comprised by the breath sample assay, an identification number associated with a specific breath of the breath sample assay (e.g., the specific breath for which information is being displayed), etc. In some instances, the session information (e.g., a session summary) may allow calibration between multiple toxin exposure assessments or may allow a technician to determine if the results of a toxin exposure assessment are unreliable. In some cases, the session information may allow a technician or operator to determine if a machine used to assay the breath sample needs to be recalibrated, repaired, or replaced.

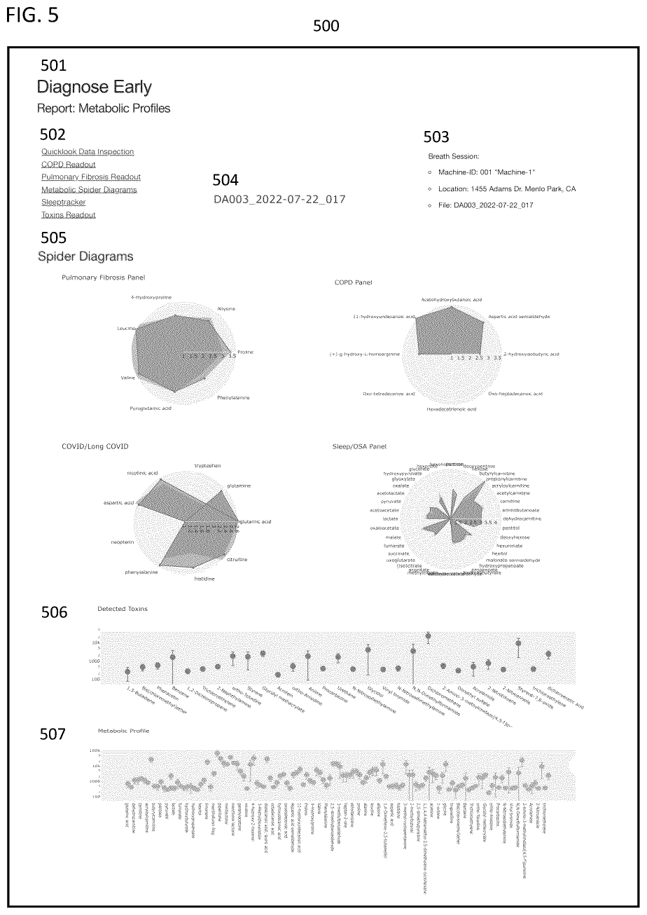

FIG. 5 provides a depiction of various metabolic profiles that may be included in a toxin exposure assessment obtained at least in part from breath assay data (e.g., a breath biopsy output file) in accordance with an embodiment of invention. Metabolic profile section 500 includes header 501 and selectable menu 502 provided to assist a viewer in navigating between sections of the toxin exposure assessment when, e.g., the assessment is displayed on an electronic viewing device (e.g., a computer or a smart phone). Session summary 503 provides information pertaining to the session in which the breath sample assay was performed. The metabolic profile breakdown further includes the identifier of the subject 504 as well as various charts and graphs depicting metabolic profiles intuitively displayed in order to, e.g., assist medical professionals with decision making. Spider diagrams 505 depict the presence and relative abundance of compounds associated with pulmonary fibrosis, COPD, COVID/long COVID, and OSA. In some instances, the shape of a spider diagram may aid in drawing conclusions, e.g., regarding potential exposure of the subject to a toxic compound or source, the diagnosis of a disease, etc. Chart 506 provides the toxins determined to be present in the breath sample obtained from the subject. Chart 507 summarizes the results of the metabolic profiles including the wide variety of various compounds determined to be present in the breath sample.

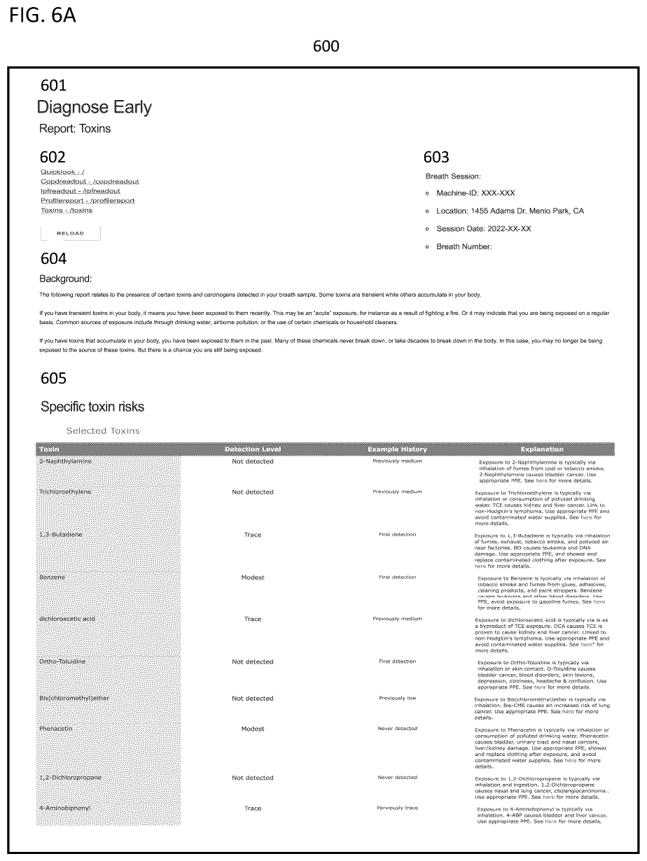

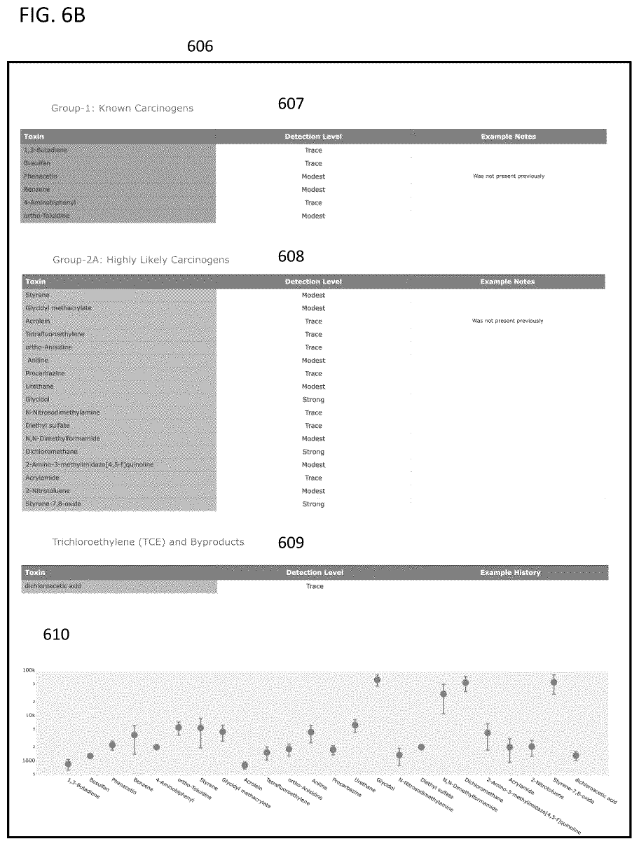

FIGS. 6A-6B provide a depiction of a toxin exposure assessment obtained from a breath sample assay in accordance with an embodiment of invention. In FIG. 6A, first page 600 of the toxin exposure assessment includes header 601 and selectable menu 602 provided to assist a viewer in navigating between sections of the assessment when, e.g., the assessment is displayed on an electronic viewing device (e.g., a computer or a smart phone). Background section 604 is provided to explain the purpose of the toxin exposure assessment to the viewer (e.g., the subject) and session summary 603 is included providing information pertaining to the session in which the breath sample assay was performed. The first page of the toxin exposure assessment further includes table 605 summarizing the findings of the toxin exposure assessment. Table 605 lists each selected toxin in a row with an assigned detection level as described above, a history of toxin presence in previous breath samples provided by the subject (e.g., as determined by the findings of one or more previous toxin exposure assessments), and an explanation regarding the toxin as described above. In FIG. 6B, second page 606 of the toxin exposure assessment breaks each selected toxin into one of tables 607-609 based on a classification of each toxin (e.g., as Group 1 or Group 2A carcinogens as discussed above). Each of tables 607-609 list selected toxins classified in the respective category in a row with an assigned detection level (e.g., as described above) and a note highlighting any changes in detected toxin level from a previous breath sample provided by the subject (i.e., a temporal change). The second page of the toxin exposure assessment further includes chart 610 summarizing the results of the breath sample assay.

### Obtaining Toxin Exposure Data

As described above, embodiments of the methods include obtaining toxin exposure data for each subject. By obtain is meant to make the exposure data accessible or available for the subsequent steps of the methods (e.g., available for training the machine learning model). In some embodiments, the exposure data may include one or more non-breath tests or assessments indicating the presence of one or more toxins in the subject's body. In some instances, the exposure data may include a history of known or potential exposures of the subject to identified sources of toxins. In some cases, the exposure data may include one or more health records.

As discussed above, embodiments of the methods include obtaining a non-breath test or assessment indicating the presence of toxins in the subject's body. In some embodiments, the non-breath test includes a blood test detecting the presence and/or amount of one or more toxins in the subject's blood. In these instances, blood may be drawn from the subject through any number of suitable means including, e.g., by using a needle or pricking the subject's finger. In some cases, the non-breath test includes a urine test detecting the presence and/or amount of one or more toxins in the subject's urine. In embodiments where the non-breath test is a blood or urine test, the blood or urine test may be performed by any number of analyzers including, e.g., any of the analyzers discussed above capable of assaying liquids. In some embodiments, the method further includes testing the subject's blood or urine for the presence and/or amount of one or more toxins.

In some embodiments, the non-breath test may include any test known to be affected by toxin exposure or, e.g., any test that may indicate one or more effects of toxin exposure on the subject's body. For example, the non-breath test may include a microbiome test, a DNA test (e.g., a DNA methylation test), or a microRNA test. In some embodiments, the exposure data may include a history of known or potential exposures of the subject to identified sources of toxins. In some cases, the history may include information regarding the subject's residence including, e.g., the source of the subject's tap water, the proximity of the subject's residence to a known toxin dumping site or manufacturing facility, how long the subject has lived in a specific residence, etc. In some instances, the history may include information regarding toxin containing products the subject is known to have used including, e.g., the duration and nature of the product's use by the subject. For example, the subject may be a dry-cleaning worker and the history may include the brand of stain removing agent used by the subject, the duration the subject used the stain removing agent, and the subject's washing and rinsing protocols involving the stain removing agent. In some cases, the history may include information regarding an employment, hobby, or activity participated in by the subject that is known to have an elevated risk of toxin exposure. For example, the history may include the amount of time the subject has been a dry-cleaning worker, fished or swam in a specific body of water, worked in a specific factory, used a specific arts and crafts product, etc.

In some cases, the exposure data may include information regarding the toxin source the subject was known or alleged to have been exposed to. In these cases, the toxin source information may include a fingerprint of the source as discussed above (e.g., the specific toxins of the source and their relative concentrations), the location and extent of the toxin source, and the duration of the toxin source (e.g., when the toxin source is an event such as a fire or chemical spill). For example, the toxin source information may include groundwater maps, the chemical makeup of degreasing or stain removing agent, the date of a train derailment and, e.g., the chemicals spilled as a result of the derailment, etc.

As discussed above, in some instances, the methods include obtaining health records for each subject. In some embodiments, the health records are associated with a disease or condition. For example, the health records may disclose the manifestation of signs or symptoms of a disease or condition in the subject. In some cases, the disease or condition may be the relative condition of the subject's overall health or the health or condition of an organ or system of the subject's body. In some instances, the disease or condition may be any disease or condition that impairs or affects the normal functioning of the body. In some instances, the disease or condition may be, e.g., an infectious disease, deficiency disease, hereditary disease, or physiological disease. In some cases, the disease or condition may result from the exposure of the subject to one or more toxins or toxin sources.

As discussed above, embodiments of the methods include obtaining a health record such as, e.g., a health record associated with a disease or condition for each subject. In some embodiments, the health record includes one or more of a personal health record (PHR), electronic medical record (EMR), or electronic health record (EHR) of the subject. In some instances, the health record includes self-reported health data such as, e.g., the subject's responses to a survey or a health information questionnaire (e.g., as described above). In some cases, the health record may include non-health data. In these instances, the non-health data may include information regarding the subject that has the potential to affect, or be affected by, the subject's health. For example, the non-health data may include one or more cohorts in which the subject belongs such as, e.g., the subject's profession, the various tasks or responsibilities associated with the subject's profession, or the location in which the subject lives or works (e.g., country, state, city, local geography, proximity to locations of interest such as, e.g., industrial facilities, etc.).

In some embodiments the health record includes one or more non-breath health assessments. While the one or more non-breath health assessments may vary, in some instances, the one or more health assessments may include a health assessment selected from the group consisting of a lung health assessment, an assessment of fitness for a given task(s), a medical imaging assessment (e.g., an ultrasound assessment), a biological sample assessment (e.g., urine tests, feces tests, blood tests, biopsies, etc.) and combinations thereof. In some instances, the biological sample assessment may include a blood panel such as, e.g., a complete blood count (CBC). In these instances, the CBC may include counts of white blood cells, red blood cells and platelets, the concentration of hemoglobin, the hematocrit, red blood cell indices, white blood cell differentials, etc. In some embodiments, the non-breath health assessment may include a microbiome test or assay (e.g., 16S sequencing or shotgun metagenomic sequencing). In some embodiments, the non-breath health assessment may include a genetic test or DNA testing.

In some embodiments, the health record may include physiological data, such as, but not limited to, one or more of heart rate, blood glucose, blood pressure, respiration rate, body temperature, blood volume, sound pressure, photoplethysmography, electroencephalogram, electrocardiogram, blood oxygen saturation, and skin conductance. In some embodiments, the physiological data may be obtained using a wearable device. Wearable devices in accordance with embodiments of the methods may include, but are not limited to, smartwatches (e.g., Apple watches, Garmin watches, or Fitbit® watches), sleep trackers (e.g., Oura rings), or heart rate monitors. In some embodiments, the wearable device may include motion sensors (e.g., accelerometers and gyroscopes), electrical sensors (e.g., electrocardiogram sensors), or light sensors (e.g., photoplethysmography (PPG) sensors). In some embodiments, the wearable device is a medical Internet of Things (IoT) device. Medical IoT devices of interest may include, but are not limited to, implanted medical devices (IMDs) (e.g., insulin pumps or defibrillators), wearable medical devices (e.g., continuous glucose monitors), and discrete devices (e.g., IoT enabled blood pressure cuffs).

In some embodiments, the non-breath health assessments and/or physiological data may be associated with the diagnosis of a disease or the assessment of a condition in the subject. In these instances, the health assessments and/or physiological data may have been used to inform the diagnosis of a disease or assess a condition in the subject. In some cases, the health assessments and/or physiological data may reflect a sign or symptom of a disease or condition in the subject. In some instances, the health assessments and/or physiological data may regard a subject diagnosed with a disease or condition or having been assessed as having a given condition of overall health, organ health, or system health (e.g., lung health is excellent, overall good, somewhat poor, overall poor, etc.). In some instances, the health assessments and/or physiological data may regard a subject known to be free of a disease or condition (e.g., the subject is healthy, the subject does not have COPD, etc.). In any of the above embodiments, the disease or condition may be associated with (e.g., known to be caused or exacerbated by) exposure to a toxin or toxin source of interest.

In some embodiments, the toxin exposure data and/or the health records may be obtained directly or indirectly from the subject, a caregiver or provider of the subject, an employer of the subject, an environmental organization, a consumer protection organization, or a database or data warehouse (e.g., as described above). In some instances, the toxin exposure data and/or the health records may be obtained, at least in part, by converting the exposure data or health records to a form compatible with a subsequent step or steps of the methods. In some embodiments, the exposure data and/or health records may be converted from a format difficult for machines to interpret to a format in a standard computer language that can be read automatically by a machine. In some cases, optical character recognition (OCR) software may be used to convert exposure data and/or health records to a form compatible with a subsequent step or steps of the methods. For example, in cases where exposure data is stored in an image format (e.g., a PDF or J PEG format), the exposure data may be converted to a JSON format, an XML format, a CSV format, a CSON format, an HTML format, etc. In these cases, organizational or categorical information structuring or classifying the exposure data and/or health records may be manually entered. For example, one or more components of a health record (e.g., as discussed above) or a section thereof, may be categorized using date or diagnosis codes (such as, e.g., diagnosis codes associated with a disease or condition). In some cases, organizational or categorical information may be automatically identified from structured digital exposure data or health records data and used to identify or classify one or more components of the exposure and/or health record data, or sections thereof, using, e.g., lines of computer code and rules-based approaches or supervised machine learning approaches paired with natural language processing software. In some instances, the exposure data and/or health records data may be obtained by scanning or imaging a plurality of records existing in hard copy form, followed optionally by conversion of the resulting image files in any of the manners discussed above.

As discussed above, embodiments of the methods include obtaining toxin exposure data for each of a plurality of subjects. In some embodiments, the exposure data may include one or more non-breath tests or assessments indicating the presence of one or more toxins in the subject's body. In some cases, the exposure data may include a history of known or potential exposures of the subject to identified sources of toxins. In some embodiments, one or more health records are obtained for each subject. The health record may include a personal health record (PHR), electronic medical record (EMR), electronic health record (EHR), self-reported health data, non-health data, non-breath health assessment and/or physiological data regarding the subject. In some cases, the toxin exposure data and/or the health record data may be provided by the subject, a caregiver or provider of the subject, an employer of the subject, an environmental organization, a consumer protection organization, or a database or data warehouse as described above. In some instances, obtaining the exposure data and/or health records may include converting the exposure data and/or health records to a form that can be read automatically by a machine and is compatible with a subsequent step or steps of the methods (e.g., automatic supervised training of a machine learning model). The toxin exposure data and/or health records obtained for each subject, together with the breath assay data generated for each subject, may then be used to train a machine learning model to identify a relationship between breath samples and toxin exposure, as discussed in greater detail below.

### Machine Learning Models

As described above, embodiments of the methods include training a machine learning model to identify a relationship between breath samples and toxin exposure using breath assay data (e.g., generated breath biopsy files) and obtained toxin exposure data. By training is meant providing or feeding the breath assay data and one or more elements of the toxin exposure data to the machine learning model so that the model can adjust one or more of its components (e.g., weights or biases) in order to or effectively (e.g., accurately or efficiently) perform a task. The machine learning model, in accordance with embodiments of the methods, may vary and may include, but is not limited to, any of the models discussed below. In some embodiments, the training may further include validating and testing.

The tasks the machine learning model is trained to perform, in accordance with embodiments of the invention, may vary. In some instances, the machine learning model may be trained to perform any task associated with assessing toxin exposure including any task demonstrated, or enabled, by the obtained toxin exposure data and/or health records as described above. For example, if the toxin exposure data provides the presence and/or concentration of one or more toxins in a subject's body, the machine learning model may be trained to determine the presence and/or concentration of one or more toxins in the subject using the subject's breath (i.e., breath assay data, e.g., a breath biopsy file generated from the subject's breath). In embodiments where the toxin exposure data reflects a known toxin exposure of a subject to a specific source of toxins (e.g., consuming contaminated drinking water), the machine learning model may be trained to indicate if a subject has been exposed to a similar source of toxins.

In some cases, the obtained toxin exposure data (e.g., as described above) is used to interpret the findings or inferences generated by a machine learning model using the subject's breath. In some embodiments, the findings or inferences generated by the machine learning model using the subject's breath may include changes in a health state or a condition of health of the subject. In these cases, the machine learning model may be trained to indicate a change in the fingerprint of a subject's breath using unsupervised machine learning techniques. For example, the subject may provide breath samples (e.g., to generate breath assay data) at two or more timepoints such that the most recent sample provided by the subject can be compared to a baseline. In some instances, the baseline may include breath sample data generated from a breath sample provided by the subject 1 day prior to the most recently provided breath sample, 1 week prior to the most recent breath sample, 1 month prior to the most recent breath sample, 6 months prior to the most recent breath sample, 1 year prior to the most recent breath sample, 5 prior to the most recent breath sample, etc. The machine learning model may then use data generated from the most recent breath sample provided by the subject and the baseline in order to look for temporal changes of the subject's breath fingerprint. The obtained toxin exposure data (including, e.g., toxin exposure data obtained at the time the baseline breath sample was provided and/or toxin exposure data obtained at the time the most recent breath sample was provided) may then be used to interpret any identified temporal changes.

In some embodiments, the tasks performed by the machine learning model may depend on the nature of the toxic compound or toxin source of interest. In embodiments where the toxin exposure data indicates the presence and/or amount of a toxin in the subject or the history of exposure of the subject to a source of toxins, the machine learning model may be trained to identify features of the toxic compound or toxin source (e.g., the relative abundance of a set of metabolites or other compounds) that correspond or correlate with exposure of the subject to the toxic compound or toxin source in order to, e.g., identify a fingerprint of the toxic compound or toxin source. The machine learning model may then be applied to breath assay data generated for a subject (i.e., separate from the breath assay data used for training) in order to indicate the presence of a toxin in the subject or the likely exposure of the subject to a source of toxins using the identified features. In embodiments where the toxin exposure data includes the subject's residence and information regarding a toxin source (e.g., a chemical spill or contaminated ground water), the machine learning model may be trained to classify breath assay data using a numerical score representative of the likelihood or severity of exposure to the toxin source the subject providing the breath sample may have.

In some embodiments, the tasks performed by the machine learning model may depend on the nature of the toxin exposure data obtained for each subject. In embodiments where the toxin exposure data includes a non-breath test used to determine the effects a toxin exposure has had on the subject, the machine learning model may be trained to identify relationships between features of a breath sample and features of the non-breath test in order to classify the breath sample as belonging to a subject having experienced certain health effects as the result of toxin exposure. For example, in instances where the toxin exposure data includes, e.g., a microbiome assessment, the machine learning model may be trained to identify features in the breath sample that correspond to the presence of specific bacteria or genes in the microbiome. The trained machine learning model may then be able to identify specific bacteria or genes in the microbiome of a subject by analyzing the subject's breath (e.g., a breath biopsy file generated from the subject's breath). In some cases, the machine learning model may be trained to only identify features in the breath sample that correspond to the presence of specific bacteria or genes in the microbiome that are indicative of toxin exposure to a specific source of toxins (e.g., using a microbiome assessment and toxin exposure data indicating the history of exposure of the subject). In some cases, the machine learning model may be trained to utilize both the toxin exposure data and obtained health records in order to identify subject's at risk for, or having, a disease or condition as the result of toxin exposure.

In some embodiments, the machine learning model be trained to identify breath assay data of insufficient quality. In these instances, bad breath assay data may be labeled (e.g., automatically or by a person of skill in the art) in order to train the machine learning model to recognize data of insufficient quality as the result of, e.g., ambient air or contamination. In some cases, the machine learning model may be trained to identify bad data (e.g., data of insufficient quality) using any of the techniques or methods used to train the machine learning model as described below (e.g., the machine learning model may be trained to determine a fingerprint for bad data).

As discussed above, the machine learning model, in accordance with embodiments of the methods, may vary and may include, but is not limited to, any of the models discussed below or any standard machine learning model, as well as combinations thereof, as is known in the art. In some embodiments, the machine learning model may depend on, e.g., the nature of the obtained toxin exposure data and the toxin(s) or source(s) of interest.

In some embodiments, the relationships between features of the breath samples and features of the toxin exposure data identified by the machine learning model may be obtained or extracted for downstream analysis. In these instances, the machine learning model may include, or be configured to employ, a linear and/or logistic regression algorithm, a linear discriminant analysis algorithm, a support vector machine (SVM) algorithm, a random forest algorithm, a K-Nearest Neighbors algorithm, a decision tree algorithm, or an XGBoost algorithm. In some embodiments, the relationships between features of the breath samples and features of the toxin exposure data identified by the machine learning model (e.g., during training) may be difficult to obtain or extract and/or may be unknown to the individuals implementing the model (e.g., the relationships may be too complexed to be understood or interpreted by a human or the relationships may contained in a component of the machine learning model considered to be a “black box”). In some instances, the features of interest (e.g., of a toxic compound or toxin source fingerprint) identified by the machine learning model and used to classify or identify components of a breath sample may not be correlated or associated with known or identified compounds. In these cases, the features of interest may include unidentified peaks or measurements (i.e., m/z signals).

In some embodiments, the machine learning model may include an artificial neural network (NN). In some embodiments, the machine learning model is a deep learning model. In these cases, the model may be three or more layers deep, such as five or more layers deep, or ten or more, or twelve or more, or thirty or more, or fifty or more, or one hundred or more. In some embodiments, the data of the breath assay data may be provided in an image format (e.g., as a total ion current (TIC) chromatogram or spider diagram). In these instances, the machine learning model may be configured to process images and may include, or be based on, a convolutional neural network (CNN), recurrent neural network (RNN), region-convolutional neural network (R-CNN), etc.

In some embodiments, the machine learning model is configured to process sequential input data. In these instances, the machine learning model may include, or be based on, a recurrent neural network (RNN) model or a transformer model. In embodiments where the machine learning model includes an RNN, the RNN may include, e.g., long short-term memory (LSTM) architecture, gated recurrent units (GRUs), or attention (i.e., may employ the attention technique or include an attention unit). In some embodiments, the machine learning model may include, or be based on, the architecture of a transformer model.

As discussed above, the machine learning model may be configured to process sequential input data. The sequential input data may be a sequence of scans presented, e.g., as temporally linked numerical matrices or images. In these instances, the machine learning model may be configured to learn from the contextual information of a scan (i.e., the scans before or after a given scan sequentially/temporally). The machine learning model may learn from the contextual information of a scan and, e.g., may learn from the past to present context of a scan and/or the present to past context of a scan. In some embodiments, the machine learning model may learn from both the past to present context and the present to left past context of a scan (i.e., the machine learning model may be bidirectional). For example, the machine learning model may include, or be based on, e.g., a bi-directional LSTM model, an RNN model with an attention, a convolutional recurrent neural network model with an attention (CRNN-A), or a transformer model. In embodiments where the bi-directional machine learning model includes, or is based on, a transformer model, the transformer model may include decoder blocks, encoder blocks and/or encoder/decoder architecture.

In some embodiments, the machine learning model is configured to select and process an individual scan of the breath assay data (e.g., breath biopsy output file). In some instances, the breath assay data may be provided as three-dimensional images (e.g., or multi-dimensional numerical matrices) aggregating or compiling data from all scans of a specific breath sample, or select scans (i.e., chosen randomly or using rules-based approaches) of a breath sample. In some embodiments, the machine learning model is configured to process negative-ion mode and positive ion mode scans separately. In other embodiments, the machine learning model is configured to process negative-ion mode and positive ion mode scans together (e.g., wherein the negative and positive mode scans are labeled accordingly). In some embodiments, the machine learning model is configured to process scans generated from different ionization agents separately. In other embodiments, the machine learning model is configured to process scans generated from different ionization agents together (e.g., wherein the scans generated while using different ionization agents are labeled with the relevant ionization agent accordingly). In some embodiments, including any of the embodiments discussed above, the machine learning model may be configured to process or analyze the breath assay data on a per breath basis.

Training may depend on, e.g., the nature or architecture of the machine learning model, the nature of the obtained toxin exposure data, and/or the nature of the toxins or sources of interest. In some cases, the machine learning model may be trained using supervised learning methods. In these cases, relevant data of interest (e.g., the presence and/or amount of toxins in a subject, the exposure of the subject to a specific source of toxins, etc.) may be extracted from the toxin exposure data obtained for each of a plurality of subject's and used to label the corresponding breath assay data of each subject. The labels or categories of interest, and the labeled breath assay data, may then be used to train the machine learning algorithm. In some embodiments, the extraction of the labels, association of the extracted labels with the generated breath assay data, and training of the machine learning model, are performed automatically using, e.g., lines of computer code and rules-based approaches or supervised machine learning approaches paired with natural language processing software. In some instances, the toxin exposure data of subjects that include relevant data of interest may be scarce. In these instances, semi-supervised learning methods may be employed. In some embodiments, unsupervised learning methods may be employed and, e.g., the categories or classifications generated by training the machine learning model may be correlated or associated with certain characteristics of patient cohorts or certain components of obtained toxin exposure data after training. In some embodiments, both supervised and unsupervised learning methods may be employed. For example, unsupervised learning methods may be used to detect any temporal changes in breath fingerprints that occur in the plurality of subjects (e.g., as described above). Characteristics of the temporal changes may then be extracted and labeled (e.g., using labels extracted from toxin exposure data) in order to train a machine learning model using supervised machine learning techniques. In this way, machine learning models may be trained to assess a subject's potential exposure to toxins using temporal changes of a subject's breath fingerprint (e.g., by comparing data obtained from a recent breath sample provided by the subject to a baseline). In other words, the exposure of the subject to a toxin source or a toxin may have a temporal change fingerprint that can be determined using machine learning techniques and utilized to assess the potential exposure of a subject.

In some embodiments, the model training algorithms and hyperparameters used to control the training may depend on, e.g., the nature or architecture of the machine learning model, the tasks the machine learning model is trained to perform, the desired accuracy or efficiency of the machine learning model, and/or the nature or size of the training data set. In some cases, the training may include methods of preventing data overfitting such as, e.g., dilution and dropout techniques.

In some cases, the training and/or the training data set (e.g., the labeled breath biopsy output files) may be modified or altered to address class imbalance. By class imbalance is meant a skewed proportion of the classes that make up a data set. For example, labeled breath assay data reflecting a specific relationship or classification (e.g., assay data labeled with an indication of the presence of one or more toxins in the subject's blood extracted from the toxin exposure data of the subject) may be relatively uncommon in the data set. In some embodiments, the training may be modified or altered to address class imbalance. For example, the optimization loss may be weighted based on class distributions. In these cases, the weighting may be learned dynamically, e.g., during training. In some embodiments, the training data set may be modified to address class imbalance. In these instances, the majority class may be undersampled. For example, in embodiments where breath assay data labeled with the presence of a toxin in the subject's blood are relatively rare, breath assay data not labeled with the presence of a toxin in blood may be randomly undersampled. In some embodiments, the majority class or classes may be randomly undersampled to achieve a ratio of one to five minority class (i.e., rare relationship or classification) to majority class(es) or less. In some instances, the majority class(es) may be undersampled to achieve a ratio of one to fifty minority class (i.e., rare relationship or classification) to majority class(es) or less, such one to twenty, or one to ten, or one to five, or one to four.

In some embodiments, the training may further include testing the trained machine learning model or machine learning models. By testing in this context is meant evaluating the trained machine learning model using labeled breath assay data different from the labeled breath biopsy data used for training after the machine learning model has finished training. In some embodiments, a first subset of the labeled breath assay data is used for training and a second subset of the labeled breath assay data is used for testing. The testing may use one or more metrics to evaluate the performance of the trained machine learning model or machine learning models. The one or more metrics may vary and may depend on the tasks performed by the trained machine learning model, the training methods employed to train the machine learning model, and the architecture of the machine learning model. For example, in embodiments where the model performs a classification task, the metric may include the number, or percent, of true positives, false positives, true negatives, or false negatives for one or more classes. In some embodiments, the metric may include a sensitivity, specificity, accuracy and/or f-score. In some instances, a metric may be determined per class. In embodiments where the metric includes an f-score, the f-score may include a macro F1-score. In embodiments where the model performs clustering or anomaly tasks, the metric may include a silhouette coefficient or any other method of evaluating an unsupervised machine learning model such as, e.g., any of the methods found in: Palacio-Nino, J., Galiano, F. B. Evaluation Metrics for Unsupervised Learning Algorithms, which are herein incorporated by reference. In some embodiments, the metric may be used to determine if the trained machine learning model performs sufficiently using, e.g., a predetermined threshold (i.e., requirement). In these instances, if the trained machine learning model does not meet the predetermined threshold, the model may be discarded and/or another model may be trained. In embodiments where another machine learning model is trained, one or more of the model architecture, training and/or the training data set may be modified prior to training. In some instances, machine learning models are trained until a trained machine learning models meets the predetermined threshold. The division between the first and second subsets of the labeled breath assay data used for training and testing, respectively, may vary. In some cases, roughly 80% of the labeled breath assay data may be used for training and roughly 20% for testing. In some instances, roughly 70% of the labeled breath assay data may be used for training and roughly 30% for testing.

In some embodiments, the training may further include validating the trained machine learning model or machine learning models. By validating in this context is meant evaluating the machine learning model during training using labeled breath assay data different from the labeled breath assay data used for training and testing. In some embodiments, a first subset of the labeled breath assay data is used for training, a second subset of the labeled breath assay data is used for testing, and a third subset of the labeled breath assay data is used for validating. The validating may use one or more metrics to evaluate the performance of the machine learning model or machine learning models such as, e.g., any of the metrics discussed above for testing. In some embodiments, the validating may be used to, e.g., select model parameters (e.g., select one or more machine learning algorithms to continue training), optimize or tune hyperparameters (e.g., model hyperparameters or algorithm hyperparameters), etc. The division between the first, second, and third subsets of the labeled breath assay data used for training, testing, and validating, respectively, may vary. In some cases, roughly 80% of the labeled breath biopsy data may be used for training, roughly 10% for testing, and roughly 10% for validating.

In some embodiments, the machine learning model is trained using the outputs from other analytical tools in addition to the breath assay data (i.e., the output from SESI-MS). In other words, both the breath assay data and data obtained using other analytical tools are used as inputs for training the machine learning model and subsequently using the trained machine learning model to perform tasks in any of the manners as described above. In some embodiments, the other analytical tools may include a Raman spectroscopy analyzer, a breathalyzer, an optical absorbance sensing analyzer, a gas chromatography analyzer, electronic sensing using an electronic nose, a nuclear magnetic resonance spectroscopy analyzer, or any of the other breath analyzers as describe above. In some cases, the other analytical tools may include analyzers configured to perform tests and generate outputs from non-breath sample provided by the subject such as, e.g., blood samples, hair sample, urine sample, DNA samples, etc.

In some cases, the machine learning model may be continuously updated based, e.g., on newly generated breath assay data and newly obtained toxin exposure data. In some instances, the machine learning model may be continuously updated based, e.g., on the data saved or archived to a database, or data warehouse, as discussed above. For example, the machine learning model may be updated by training incrementally as new data comes in, in batches once a certain amount of new data is available, or the machine model may be retrained from scratch once a certain amount of new data is available. In some cases, the machine learning model may be updated incrementally or in batches, and then completely retrained once a certain amount of new data is available (e.g., every certain number of batch updates).

As discussed above, embodiments of the methods include training a machine learning model to identify a relationship between breath samples and toxin exposure using generated breath assay data and obtained toxin exposure data. In some embodiments, the relationship may be difficult to obtain or extract (e.g., the relationship may be too complexed to be understood or interpreted by a human or the relationships may contained in a component of the machine learning model considered to be a “black box” such as within multiple layers of a NN). The machine learning model may be trained to perform any task associated with assessing the potential exposure of a subject to toxins including any task demonstrated, or enabled, by the obtained toxin exposure data and/or health records as described above. For example, the machine learning model may be trained to identify relationships between features of a breath sample (e.g., identified or unidentified m/z peaks) and the exposure of a subject to a source of toxins. The machine learning model may include, but is not limited to, any of the discussed models or any standard machine learning model, as well as combinations thereof, as is known in the art.

In some embodiments, the machine learning model may include an artificial neural network (NN). For example, the machine learning model may include, or be based on the architecture of a recurrent neural network (RNN) or a transformer model. Training may depend on, e.g., the nature or architecture of the machine learning model, the nature of the obtained toxin exposure data, and/or the nature of the toxic compound or toxin source of interest. In some cases, the machine learning model may be trained using supervised learning methods and relevant data of interest (e.g., the presence of toxins in a subject's blood or urine, known exposures of a subject to a source of toxins) may be extracted from the toxin exposure data and used to label the corresponding breath assay data of each subject. In some instances, the machine learning model may be trained using unsupervised approaches. In some cases, both supervised and unsupervised approaches may be utilized in order to assess the potential exposure of a subject to toxins. In some embodiments, the training may further include validating, and testing of the machine learning model. The trained machine learning model may be applied to breath assay data (e.g., different from the data used for training) to generate a toxin exposure assessment (e.g., as discussed above) and a health evaluation, as discussed in greater detail below.

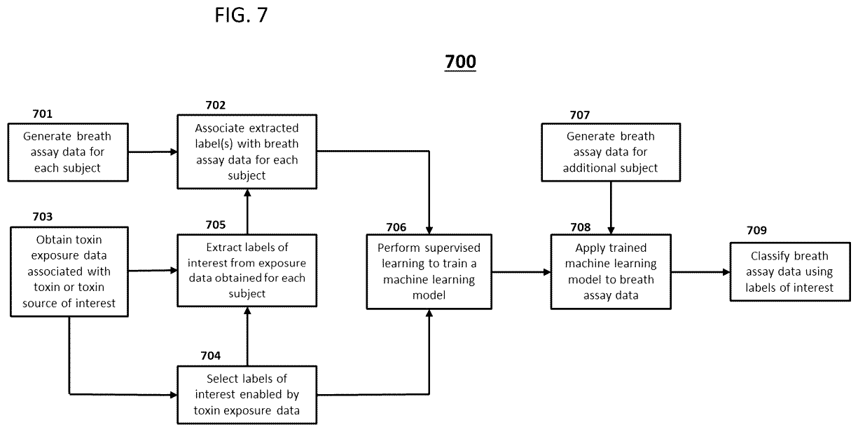

FIG. 7 provides a flow diagram depicting a method for training a machine learning model using generated breath assay data and obtained toxin exposure data in accordance with an embodiment of the invention. At step 701, breath samples from a plurality of subjects are analyzed with a secondary electrospray ionization-high-resolution mass spectrometry analyzer to generate breath assay data for each subject. At step 703, toxin exposure data associated with a toxic compound (e.g., TCE or a metabolite thereof) or toxin source of interest is obtained for each subject. The obtained toxin exposure data is then used to select labels of interest enabled by the exposure data at step 704, and extract the labels of interest from the exposure data associated with each subject at step 705. At step 702, the extracted labels (e.g., the results of a urine or blood test or the circumstances of a toxin exposure) are then associated with the breath assay data corresponding to the patient for which the toxin exposure data used to extract each label was obtained. At step 706, a machine learning model (such as, e.g., a RNN, CNN, transformer, or regression model) may then be trained using supervised or semi-supervised machine learning methods, the labeled breath assay data, and the selected labels of interest. In some embodiments, components of the obtained health records such as, e.g., other non-breath health assessments or physiological data, are also labeled and used to train the machine learning algorithm along with/in addition to the labeled breath assay data. At step 707, additional breath assay data, separate from the breath assay data used for training, is generated from a subject. At step 708, the train machine learning algorithm is applied to the breath assay data in order to classify the breath assay data (step 707). In some cases, the breath assay data is classified as, e.g., generated by a subject having a certain amount of a toxin in their body, having been exposed to a specific source of toxins, etc. The classified breath assay data, along with any other toxin exposure data and/or health records obtained for the subject, may then be saved to a database or a data warehouse (e.g., as discussed above) in order to continuously train the machine learning model or train other machine learning models that may be applied to future breath assay data.

### Generating a Health Evaluation

As discussed above, embodiments of the invention include applying a trained machine learning model to breath assay data in order to generate a toxin exposure assessment regarding potential toxin exposure for a subject or subjects. The breath assay data may then be used to generate a health evaluation for the subject. The health evaluation is a qualitative or quantitative determination regarding one or more health related matters pertaining to the subject. The health evaluation, generated in accordance with embodiments of the methods, may vary. In some embodiments, the health evaluation may be generated for the subject from breath assay data such as, e.g., one or more exposure assessments generated as discussed above. In some embodiments, the health evaluation may be generated or obtained based at least in part on one or more exposure assessments as discussed above and/or on health records obtained from the subject (e.g., data not obtained from a breath sample).

A health evaluation may be generated or obtained at two or more timepoints. In some instances, a health evaluation may be generated or obtained at three or more timepoints (e.g., to generate three or more health evaluations, such as four or more, or five or more, or ten or more). The two or more timepoints may be at least a day apart from each other, such as at least a week apart from each other, or at least a month apart from each other, or at least a year apart from each other. In some instances, a first timepoint of the two or more timepoints may occur after a potential exposure of the subject to a source of toxins. In other cases, a first timepoint of the two or more timepoints occurs before a potential exposure of the subject to a source of toxins in order to, e.g., function as a baseline as discussed above. In these instances, the first timepoint may occur prior to the subject initiating employment (e.g., as a firefighter, cleaner, industrial worker, etc.) or moving to a new location. The subject may be assayed (i.e., a timepoint may occur) every set number of days or months while they are at a certain location or working a certain profession (e.g., firefighting, dry cleaning).

As described above, the health evaluation may include breath assay data (i.e., one or more exposure assessments) and non-breath assay data (i.e., a health record as described above including, e.g., other health assessments, the subject's medical history, data gathered from wearable devices, etc.). In some instances, the health evaluation includes an interpretation of the breath assay data and non-breath assay data. The interpretation may be derived based on the breath assay data and non-breath assay data either individually and/or in combination with one another. In some embodiments, the interpretation may include the likelihood that the subject has a disease or condition (e.g., a potential diagnosis). In these instances, the interpretation may include the severity or stage of the disease or condition. In some embodiments, the interpretation may include the likelihood or risk level the subject may have of developing a disease or condition. In some cases, the presence of one or more toxic compounds (determined directly or indirectly) and the abundance (e.g., concentration or relative intensity) of each compound relative to one another in a breath sample may be correlated with a disease or condition. In other words, the fingerprint of a disease or condition detectable in a subject's breath may include one or more toxic compounds (or, e.g., may include similar compounds to a toxin compound or source fingerprint). Alternately, the fingerprint may be a single or series of highly correlated compounds that may have a known or unknown causal correlation to the disease or condition other than its high level of correlation, whether the correlation is found by basic analysis or complex Al modeling. In some embodiments, the likelihood or risk level the subject may have of developing a disease or condition is generated by analyzing or assaying the breath sample for the presence of one or more toxic compounds of a disease or condition fingerprint. In these cases, the likelihood or risk level the subject may have of developing a disease or condition may be generated by comparing the fingerprint of the disease or condition to the toxic compounds detected in the breath sample assay as indicated by one or more exposure assessments.

As described above, the health evaluation may include an interpretation of the breath assay data alone or in combination with non-breath assay data. This interpretation may include a potential diagnosis and/or a risk level of developing a disease or condition generated by comparing the fingerprint of a disease or condition to the determined presence of one or more toxic compounds of the disease or condition fingerprint (e.g., and the values of abundance thereof) in the breath sample. For example, a risk level for developing a cancer such as, e.g., kidney cancer, can be generated by comparing the determined presence of one or more toxic compounds in the breath sample to toxic compounds associated or correlated with kidney cancer when found in breath (i.e., a determined kidney cancer fingerprint of toxic compounds or metabolites). In some embodiments, the correlation or association of toxic compounds found in a breath sample to a specific disease or condition (i.e., the relationship between toxic compounds found in a breath and a disease or condition) is determined using previously generated exposure assessments. For example, the correlation or association can be determined by comparing the exposure assessments generated by breath samples of healthy patients with the exposure assessments generated by the breath samples of patients diagnosed with a disease or condition. In some cases, the correlation or association may be generated using a machine learning algorithm (e.g., by training a machine learning model).

As discussed above, the presence of one or more toxic compounds and the abundance of each compound relative to one another in a breath sample (e.g., indicated by one or more exposure assessments) may be used to generate a risk level of a subject to a disease or condition. In some embodiments, the one or more exposure assessments may be used to inform a risk level or likelihood of developing a disease or condition, e.g., along with non-breath assay data as discussed above (e.g., microbiome tests, wearable device data, the subject's medical history, etc.). For example, the one or more exposure assessments may be paired with wearable device data to help determine a risk level or likelihood the subject may have of developing a disease or condition.

As discussed above, the health evaluation may include an interpretation derived based on the breath and non-breath assay data either individually and/or in combination with one another. In some embodiments, the interpretation may include a general assessment of the subject's overall health or the health or condition of an organ or system of the subject's body. For example, the interpretation may include a general assessment of the subject's lung health (e.g., lung health is excellent, overall good, somewhat poor, overall poor, etc.). In some embodiments, the interpretation may include a general assessment of a specific category of health risk or health threat to the subject. For example, the interpretation may include a general assessment regarding the threat or risk of toxin exposure to the subject. In some embodiments, the interpretation may include a general assessment of a subject's fitness for performing a task (e.g., driving, running, etc.) or undertaking a duty or responsibility (e.g., firefighting, piloting a vehicle, policing, construction, manufacturing, etc.). By “fitness” is meant the ability of the subject to perform and/or the risks associated with the subject undertaking (e.g., the potential risks to themselves, others, property, etc.) a task or tasks associated with the duty or responsibility. For example, the interpretation may include a general assessment regarding the fitness of a firefighter for duty.

In some embodiments, the health evaluation may include a suggested next course of action. In embodiments where a next course of action is suggested, the suggested course of action may vary. In some instances, the course of action includes obtaining additional tests or consulting with additional medical professionals. For example, the suggested course of action may include consulting a specialist wherein a secondary opinion may be obtained, or additional testing may be recommended or ordered. In some embodiments, the suggested course of action may include a temporary or permanent modification to the subject's responsibilities of employment. For example, the suggested course of action may include a period of time wherein the subject should avoid any potential for smoke inhalation if the subject is, e.g., a firefighter. In some embodiments, the suggested course of action may include an explanation regarding typical manners in which an individual may be exposed to a toxic compound (e.g., sources of the toxins) and steps the subject may take to avoid potential future exposure to toxins. For example, the suggested course of action may include preventative measures, such as, e.g., recommended personal protective equipment (PPE) or specific protocols for washing PPE. In some embodiments, the suggested course of action may include a potential treatment regimen or therapy recommendation. By treatment regimen is meant a treatment plan that specifies the quantity, the schedule, and the duration of treatment. For example, the treatment regimen may include a suggested drug regimen, a detoxification process (e.g., blood/plasma donation), or a suggested lifestyle change (e.g., dietary or exercise plans, etc.).

In some instances, the health evaluation may include an evolution of a health risk, a disease or condition severity, or a likelihood of developing a disease or condition. By “evolution” is meant a progression of a metric over time, e.g., the progression of a health risk, a condition or disease severity, or a likelihood of developing a disease or condition over time. In some cases, the evolution is generated based at least in part on one or more previously obtained health evaluations or exposure assessments. In some embodiments, the health evolution includes an explanation of how the relevant metric has changed over time. For example, the health evolution may include a peak, periods of decline or incline, and whether the metric is in a period of incline or decline at the time the present health evaluation was obtained. In some embodiments, the health evolution may include an assessment of the effectiveness of a previously suggested next course of action (e.g., as described above). For example, the health evolution may include an assessment of the effectiveness of previously suggested preventative measures or detoxification processes. The assessment of effectiveness may be obtained based on whether the health evolution indicates the level of potential exposure to a toxic compound (e.g., a potential health risk) is in a period of incline or decline at the time the present health evaluation was obtained.

In some embodiments, the health evaluation may include one or more health scores. By health score is meant a quantitative evaluation of the subject's overall health, the health or condition of an organ or system of the subject's body, a health risk facing the subject, or the subject's fitness for performing a task or undertaking a duty or responsibility compared with a baseline. The baseline may vary, and in some instances includes the average of data associated with a cohort, such as an average level or amount of a given toxic compound found in a population or cohort of interest, or a likelihood of developing a disease or condition in a population or cohort of interest. In some instances, the baseline includes prior data obtained from the subject, e.g., prior data obtained from the subject 1 day prior to generating the health evaluation, 1 week prior to generating the health evaluation, 1 month prior to generating the health evaluation, 6 months prior to generating the health evaluation, 1 year prior to generating the health evaluation, 5 years prior to generating the health evaluation, etc. In some embodiments, a health score is generated for the subject's overall health, lung health, exposure to toxins, risk of developing a disease or condition, or fitness for the duty associated with their employment (e.g., firefighting). The health score may be generated or obtained using breath assay data and/or non-breath assay data as discussed above. For example, an overall health score may be generated that is a combination of the one or more exposure assessments (e.g., as discussed above) and one or more additional health assessments.

In some instances, the health evaluation may include one or more personalized insights. A personalized insight may vary and includes, but is not limited to, the detection of an anomaly, a classification, the detection of a cluster, or a forecast. In some instances, the personalized insight includes an insight regarding the subject individually. In other instances, the personalized insight includes an insight regarding a group or cohort in which the subject belongs. In embodiments where the insight includes the detection of an anomaly, the insight may include the identification of unusual data. For example, the insight may be that a specific toxic compound (e.g., TCE) is detected at a higher level or concentration than usual or the risk of developing a disease or condition is elevated (e.g., when compared to a baseline as described above). In embodiments where the insight includes a classification, the insight may include the identification of a group with similar data to the subject and, e.g., assigning and comparing the results and/or data of the subject to the group. For example, the insight may be that the subject has better overall health than 70% of firefighters (e.g., when the subject is a firefighter). In embodiments where the insight includes the detection of a cluster, the insight may include finding groups with similar results. For example, the insight may be that a city or location has the highest rate of exposure to one or more toxic compounds.

As discussed above, the health evaluation may include one or more personalized insights. In some embodiments, the personalized insight may include a forecast. The forecast may include an evolution of a health risk, a disease or condition severity, or a likelihood of developing a disease or condition as described above. In some embodiments, the forecast may include an evolution of the subject's overall health, the health or condition of an organ or system of the subject's body, or the subject's fitness for performing a task or undertaking a duty or responsibility compared with a baseline. In some embodiments, the forecast may include a predicted future outcome such as, e.g., a health outcome prediction for the subject. The health outcome can be predicted at least in part using a health evaluation and/or an exposure assessment obtained as discussed above. For example, the predicted health outcome may be that the subject has a high risk of developing a specific disease or condition (e.g., COPD or a specific form of cancer). In some instances, the health outcome can be predicted at least in part using a dynamic algorithm. The dynamic algorithm may be a machine learning algorithm, such as a machine learning algorithm that uses an artificial neural network. In some instances, the health evaluation is used to determine if a particular event or source of toxin exposure has affected the subject's predicted health outcomes. In instances where the subject is assayed at two or more timepoints to generate two or more health evaluations and/or exposure assessments the two or more health evaluations and/or exposure assessments may be used to, e.g., determine changes in exposure of the subject to toxins over time, determine a clearance time of toxins from the subject, or predict one or more health outcomes for the subject using some combination of the two or more health evaluations and/or exposure assessments. In some cases, some combination of the two or more health evaluations and/or exposure assessments is used to determine if a particular event or source of toxin exposure has affected the subject's predicted health outcomes.

In some embodiments, the health evaluation may include notes or explanations aiding the subject, or a person associated with the subject, in interpreting the results of the health evaluation. For example, the evaluation may include an explanation regarding typical manners in which an individual may be exposed to a toxic compound (e.g., sources of the toxin), steps the subject may take to avoid potential future exposure to the toxic compound, and/or diseases and conditions associated with exposure of an individual to the toxic compound. In some cases, the health evaluation may include notes indicating information relevant specifically to the subject. For example, the evaluation may include a note indicating a difference between the level of one or more toxic compounds in the breath sample relative to a baseline, or symptoms associated with the determined level of one or more toxic compounds in the breath sample (e.g., dizziness, headaches, skin lesions, etc.). In some embodiments, the health evaluation may include a background section such as, e.g., a background section explaining the purpose of various exposure assessments (generated, e.g., as discussed above). In some embodiments, the health evaluation may include visual means aiding the subject, or a person associated with the subject, in interpreting the findings of the health evaluation and/or the one or more exposure assessments as discussed above (e.g., figures, charts, images, etc.). For example, in embodiments where the health evaluation includes an exposure assessment, a figure including a number line with the relevant ranges displayed and an indication of the subject's score may accompany the exposure assessment. In some embodiments, the health evaluation may include a metabolic profile or metabolic profiles of the breath sample of the subject as described above. In these instances, a spider diagram may be provided for each metabolic profile.

The health evaluation may include information pertaining to the session in which the breath sample assay was performed (e.g., the session from which one or more exposure assessments were generated). For example, the health evaluation may include, e.g., the date in which the breath sample assay was obtained, an identification number associated with a machine used to assay the breath sample, the location in which the breath sample assay took place (e.g., the address of a pharmacy, clinic, hospital, or primary care physicians office), the number of breaths comprised by the breath sample assay, an identification number associated with a specific breath of the breath sample assay (e.g., the specific breath for which information is being displayed), etc. In some instances, the session information (e.g., a session summary) may allow calibration between multiple exposure assessments and/or health evaluations or may allow a technician to determine if the results of an exposure assessment and/or a health evaluation are unreliable. In some cases, the session information may allow a technician or operator to determine if a machine used to assay the breath sample needs to be recalibrated, repaired, or replaced.

As discussed above, a health evaluation may be generated for a subject from breath assay data (e.g., one or more exposure assessments) or non-breath assay data. In some instances, the health evaluation is generated in real time. By “real time” is meant the health evaluation is generated during or immediately following the breath sample assay (e.g., during collection of the breath sample or while the breath sample is being assayed using, e.g., a mass spectrometer). In some instances, the health evaluation is generated in two hours or less. In some cases, the health evaluation is generated in one hour or less, such as thirty minutes or less, or twenty minutes or less, or ten minutes or less, or five minutes or less, or one minute or less following the breath sample assay. In some instances, the health evaluation is generated in real-time using a brief biopsy output file, e.g., as described in U.S. Provisional Application Ser. Nos. 63/359,134 and 63/416,185 (Attorney docket nos. DIAG-003PRV and DIAG-003PRV2, respectively); the disclosures of which are herein incorporated by reference.

In some instances, the health evaluation is generated or obtained using a dynamic algorithm. The dynamic algorithm may be a machine learning algorithm, such as a machine learning algorithm that uses an artificial neural network. In some embodiments, the health evaluation is generated by training a machine learning model such as, e.g., any of the models as discussed above. In some instances, the health evaluation (e.g., obtained or generated as discussed above) is associated with an identifier of the subject as discussed above. In some instances, the health evaluation and associated identifier may be saved via local storage and/or cloud storage and, e.g., may be saved to a database such as a data warehouse. In some embodiments, the health evaluation may be generated based on correlations and relationships determined from previously saved health evaluations and/or exposure assessments such as, e.g., the health evaluations and/or exposure assessments saved to a data warehouse. These previously saved health evaluations and/or exposure assessments may include health evaluations and/or exposure assessments generated from the subject presently obtaining the health evaluation and/or generated from other subjects for which health evaluations were previously obtained. For example, a data warehouse containing previously generated health evaluations and/or exposure assessments may be used to determine a relationship between toxin exposure and health outcomes such as, e.g., a relationship between toxin exposure and the risk level or likelihood of a subject developing a disease of condition. The determined relationship may then be used to generate a subsequent health evaluation. In some embodiments, the relationship or correlation may be determined, at least in part, using a dynamic algorithm. The dynamic algorithm may be a machine learning algorithm, such as, e.g., a machine learning algorithm using a neural network. In some embodiments, the relationship or correlation is determined by training a machine learning model such as, e.g., any of the models as discussed above. In some instances, the determined relationship may be used to generate a subsequent health evaluation.

In some embodiments, the method further includes suggesting preventative measures based on the health evaluation, such as, e.g., recommended personal protective equipment (PPE) to avoid potential future exposure to a toxic compound or the development of a disease or condition. In some embodiments, the method further includes providing a therapy recommendation to the subject based on the health evaluation. While the therapy recommendation may vary, in some instances the therapy recommendation includes recommendations regarding the specifics of administering some existing standard of care for the treatment of a disease or condition such as, e.g., a detoxification regime (e.g., blood/plasma donation). In some instances, the method further includes administering the treatment to the subject.

Embodiments of the methods may further include transmitting the health evaluation and/or one or more exposure assessments, e.g., to a health care practitioner, to the subject, to an agent of the subject, etc. In some instances, the health evaluation and/or one or more exposure assessments are received by a computer or mobile device application, such as a smart phone or computer app. In some cases, the health evaluation and/or one or more exposure assessments are received by mail, electronic mail, fax machine, etc. Aspects of the invention further include methods of obtaining a health evaluation and/or one or more exposure assessments, e.g., by breathing into a system of the invention as discussed in greater detail below; and receiving a health evaluation and/or one or more exposure assessments from the system.

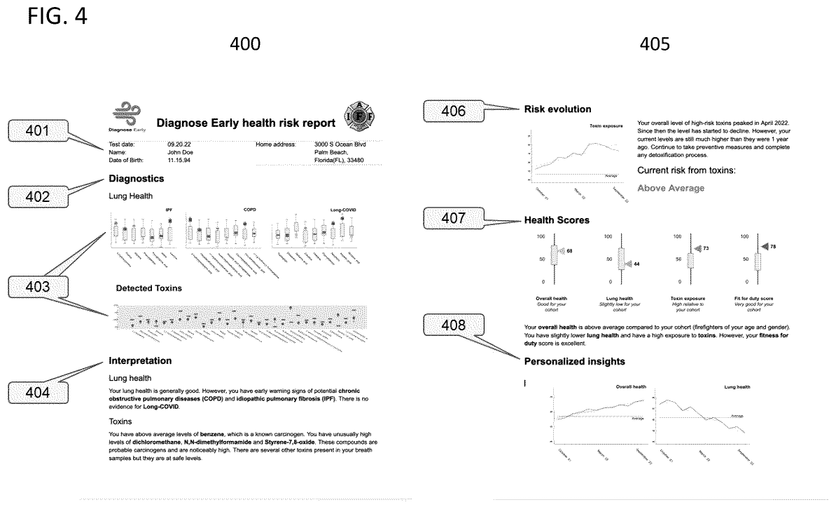

FIG. 4 provides a depiction of a health evaluation obtained at least in part from a breath sample assay in accordance with an embodiment of invention. In FIG. 4, first page 400 of the health evaluation includes header 401 including information pertaining to the session in which the health evaluation was generated and identifying information of the subject. Diagnostics section 402 includes breath assay data 403 including a chart summarizing results of a toxin exposure assessment and a chart depicting compounds detected in the breath assay associated with various diseases or conditions. Following diagnostics section 403, interpretation section 404 explains the significance of the breath assay data (and, e.g., the non-breath assay data) on the subject's lung health and the health risks toxins may pose to the subject. The second page 405 of the health evaluation includes toxin health risk evolution 406 and various health scores 407 obtained, e.g., as described above. Personal insights 408 are also provided as charts depicting evolutions of the subject's overall health and lung health over the previous year and up to the present timepoint the depicted health evaluation was obtained.

### Systems

Aspects of the present disclosure further include systems, such as computer-controlled systems, for practicing embodiments of the above methods. Aspects of the systems include: a particle analyzer configured to receive a breath sample; a processor configured to receive the measurements generated by the particle analyzer; and memory operably coupled to the processor wherein the memory includes instructions stored thereon, which when executed by the processor, cause the processor to determine if the subject has been exposed to one or more toxins or toxin associated compounds (e.g., TCE and/or one or more metabolites thereof). In some cases, the memory may include instructions stored thereon, which when executed by the processor, cause the processor to generate or obtain a toxin exposure assessment that can be used to determine if the subject has been exposed to a toxin and to monitor changes in toxin exposure over time in the subject using a machine learning model. In certain embodiments, the memory may include instructions stored thereon, which when executed by the processor, cause the processor to: analyze breath samples from a plurality of subjects with a secondary electrospray ionization-high-resolution mass spectrometry analyzer to generate breath assay data; obtain toxin exposure data for each subject; train a machine learning model to identify a relationship between the breath samples and toxin exposure using the breath assay data and the obtained toxin exposure data; and apply the trained machine learning model to breath assay data, different from the breath assay data used to train the model, to generate an exposure assessment regarding potential toxin exposure for a subject or subjects. In some embodiments, the memory may include instructions stored thereon, which when executed by the processor, cause the processor to generate a health evaluation using the toxin exposure assessment and, e.g., one or more health records obtained for each subject.

In some embodiments, the particle analyzer may be a mass spectrometer. The mass spectrometer may be configured to perform a variety of techniques/methods. In some embodiments, the mass spectrometer includes a high-resolution mass spectrometer (HRMS). In some embodiments, the mass spectrometer may be coupled to or include one or more of: an ion mobility spectrometer (IMS), a gas chromatograph (GC), a liquid chromatograph (LC), a differential mobility spectrometer (DMS), a field asymmetric ion mobility spectrometer (FAIMS), a selective-ion flow tube (i.e., SIFT-MS), a proton-transfer-reaction (i.e., PTR-MS), a time-of-flight mass spectrometer (TOF-MS) etc. In some embodiments, the mass spectrometer may be a Thermo Scientific high-resolution mass spectrometer (e.g., Thermo Scientific Exactive™, Q-Exactive™ Exploris™) or a SciEX high-resolution mass spectrometer (e.g., TripleTOF® mass spectrometer system).

In order for a mass spectrometer to measure the mass to charge ratio of a particle or particles (e.g., toxins), the particle(s) must first be ionized or charged using, e.g., an ionizer. The ionizer (e.g., ionization source) coupled to the mass spectrometer in accordance with embodiments of the invention may vary. In some embodiments, the ionizer is configured to perform matrix-assisted laser desorption/ionization (MALDI), atmospheric pressure chemical ionization (APCI), atmospheric pressure photoionization (APPI), electrospray ionization (ESI), secondary electrospray ionization (SESI), etc. In some embodiments, the ionizer is configured to perform SESI. In these instances, the ionizer may be a SUPER SESI™ device (e.g., a SUPER SESI™ QE or SUPER SESI™-X device). The ionizer may be configured to ionize particles in the breath sample, wherein the mass spectrometer may be configured to generate measurements of the mass-to-charge ratio of the ionized particles.

In some embodiments, the mass spectrometer is configured to provide real-time feedback of the breath sample assay related to the quality of the breath sample. In some embodiments, the ionizer and mass spectrometer are configured to assay the breath sample in real time with respect to the subject providing the breath sample. In these embodiments, compounds that are exhaled from deeper in the lungs may be detected relatively later in the assay. In some embodiments, the mass spectrometer is configured to measure the time of detection of a compound (e.g., a toxin) in the breath sample assay.

The systems may further include means for delivering a breath sample (e.g., one or more exhaled breaths of the breath sample) from the subject to the particle analyzer. In some instances, these delivery means may include a mouthpiece configured to seal to the lips of a subject and receive the breath sample from the subject. The delivery means may additionally include a breath chamber configured to receive the breath sample from the mouthpiece. In some instances, the breath chamber is operably coupled to the ionizer. In these cases, the delivery means may further include a valve configured to do one or more of: direct the breath sample along a desired flow path, control the flow rate of the breath sample into the ionizer, or block the flow of ambient air/the breath sample. In other cases, the breath chamber is configured to produce exhaled breath condensate (EBC) from the breath sample. In these instances, the system may include means for chilling the breath chamber. Chilling means may include, but are not limited to, a freezer or refrigerator, dry ice, or liquid nitrogen. In embodiments where an ECB is used, the system may further include aerosolization means configured to aerosolize the EBC prior to ionization such as, e.g., a nebulizer. In embodiments where an ECB is used, the system may further include means for stably storing the EBC such as, e.g., a refrigerator or a freezer.

In some embodiments, the memory includes instructions stored thereon, which when executed by the processor, further cause the generate breath assay data for a plurality of subjects and obtain toxin exposure data for each subject according to any of the methods as discussed above. In these instances, the instructions, when executed by the processor, may cause the processor to train a machine learning model to identify a relationship between the breath samples and toxin exposure using the breath assay data and the obtained toxin exposure data according to any of the methods as discussed above.

In some embodiments, the memory includes instructions stored thereon, which when executed by the processor, further cause the processor to generate a exposure assessment according to any of the methods as discussed above. In some embodiments, the memory includes instructions stored thereon, which when executed by the processor, further cause the processor to generate a health evaluation according to any of the methods as discussed above. In these instances, the instructions, when executed by the processor, may cause the processor to first generate the toxin exposure assessment before generating the health evaluation based, at least in part, on the exposure assessment.

In some embodiments, the processor includes instructions stored thereon, which when executed by the processor, further cause the processor to generate an identifier associated breath biopsy output file according to any of the methods as discussed above. In some embodiments, the processor includes instructions stored thereon, which when executed by the processor, further cause the processor to generate an intuitive data set based on the identifier associated breath biopsy output file according to any of the methods as discussed above. In these instances, the instructions, when executed by the processor, may cause the processor to reduce the data of the identifier associated breath biopsy output file in order to generate an intuitive data set according to any of the methods as discussed above. In some embodiments, the instructions, when executed by the processor, may cause the processor to first generate the identifier associated breath biopsy output file before generating the toxin exposure assessment and/or the health evaluation according to any of the methods as discussed above.

In some embodiments, the processor includes instructions stored thereon, which when executed by the processor, further cause the processor to dynamically adjust breath collection automatically based on real-time feedback according to any of the methods as discussed above.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-ft magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C #, C++, Go, R, Swift, PHP, as well as any many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to determine if a subject has been exposed to a toxin or toxin source. In some embodiments, the methods and systems described herein find use when it is desirable for any potential toxin exposure to be rapidly determined and/or determined from non-invasively obtained samples. In some embodiments, the methods and systems described herein find use in applications where mass testing of a cohort or group for toxin exposure is desired. In some embodiments, the methods and systems described herein find use in applications where it is desired to determine the impact of a potential source of toxins on a cohort or group of interest. Embodiments of the present disclosure find use in applications where it is desired to test a subject for toxin exposure at a low cost, with a short turnaround time, or with high performance (e.g., reproducibility, accuracy, and precision). In some embodiments, the subject methods and systems may facilitate toxin exposure testing of a subject by low/minimally trained technicians. In some embodiments, the subject methods and systems may facilitate diagnosis for one or more conditions, insight on one or more health risks, or recommendations for one or more therapies or treatments.

### Examples

The following examples are put forth so as to provide those of ordinary skill in the art with a complete disclosure and description of how to use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### I. Detection of Toxins in Breath

Group 1 Carcinogens

The breath sample of a healthy subject was assayed for the presence of twelve Group 1 carcinogens. The breath sample assay was run using a high-resolution mass spectrometer coupled to a SUPER SESI™ device run on negative-ion mode. The numbers reflect a value of abundance and a “—” denotes non-detection. Non-detection indicates that the respective compound was not present in the breath sample above the limit of detection. Most toxins are detected at trace levels, some in only one or two of the five breaths assayed. The results of the breath sample assay appear in Table 3, below:

TABLE 3
Detection of Group 1 carcinogens

| Candidate compound | Breath 1 | Breath 2 | Breath 3 | Breath 4 | Breath 5 |
|---|---|---|---|---|---|
| 1,3-Butadiene | 252.63 | 175.69 | 160.99 | 159.71 | 155.42 |
| Chlornaphazine | — | — | — | 503.01 | — |
| Thiotepa | — | — | — | 261.71 | — |
| Bis(chloromethyl)ether | 454.85 | 530.18 | 485.32 | 584.33 | 519.75 |
| Phenacetin | 563.61 | 505.41 | 579.25 | 364.82 | 470.61 |
| Benzene | 1,554.04 | 467.25 | 342.31 | 555.29 | 566.49 |
| Vinyl Chloride | — | — | 149.12 | 95.48 | — |
| 1,2-Dichloropropane | 270.83 | 264.36 | 386.38 | 454.11 | 356.73 |
| Trichlorethylene | 356.71 | 210.52 | — | — | — |
| 2-Napthylamine | 475.89 | 491.23 | 482.98 | 405.22 | 441.65 |
| 4-Aminobiphenyl | 393.12 | — | — | 218.55 | — |
| Ortho-Toluidine | 1,776.38 | 1,014.45 | 1,041.97 | 1,124.91 | 1,083.01 |

Group 2 Carcinogens

The breath sample of a healthy subject was assayed for the presence of thirty-two Group 2 carcinogens. The breath sample assay was run using a high-resolution mass spectrometer coupled to a SUPER SESI™ device run on negative-ion mode. The numbers reflect a value of abundance and a “—” denotes non-detection. Non-detection indicates that the respective compound was not present in the breath sample above the limit of detection. Most toxins are detected at trace levels, some in only one or two of the five breaths assayed. Some toxins are not detected in any breaths of the breath sample assay, which may indicate a high elimination rate of the toxin in the human body or a limited exposure of the subject to the toxin. The results of the breath sample assay appear in Table 4, below:

TABLE 4
Detection of Group 2 carcinogens

| Candidate compound | Breath 1 | Breath 2 | Breath 3 | Breath 4 | Breath 5 |
|---|---|---|---|---|---|
| Epichlorohydrin | 205.000 | — | — | 165.674 | — |
| Glycidyl methacrylate | 2538.123 | 2334.068 | 2381.556 | 1874.924 | 1942.170 |
| Ethylene dibromide | — | — | — | — | — |
| Acrolein | 174.874 | 157.883 | 131.375 | 111.137 | 113.003 |
| 1,3-Propane sulfone | — | 185.946 | 188.197 | 200.619 | — |
| Tetrafluoroethylene | — | — | — | — | 189.519 |
| CCNU | — | 279.934 | — | — | — |
| ortho-Anisidine | 498.895 | 349.002 | 432.636 | 346.644 | 419.485 |
| Aniline | 1753.159 | 822.934 | 813.784 | 1031.548 | 1120.484 |
| 2-Mercaptobenzothiazole | 408.572 | 280.439 | 272.228 | — | — |
| Indium phosphide | 203.526 | — | — | — | — |
| Chloral hydrate | 464.357 | 327.259 | — | 295.967 | 253.122 |
| Procarbazine | 374.553 | 327.719 | 266.784 | 287.853 | 233.682 |
| Urethane | 1588.290 | 848.862 | 965.777 | 1548.113 | 1429.056 |
| 1,2-Dimethylhydrazine | — | — | — | 107.105 | — |
| N-Nitrosodiethylamine | 352.730 | 422.175 | 317.049 | 362.179 | 431.367 |
| Glycidol | 3919.651 | 1188.937 | 957.200 | 817.743 | 678.698 |
| Vinyl bromide | 338.814 | 249.767 | 277.850 | 344.196 | 236.513 |
| N-Nitrosodimethylamine | 392.530 | 251.034 | — | 352.236 | 242.767 |
| N,N-Dimethylformamide | 3359.046 | 684.792 | 568.697 | 998.142 | 1123.350 |
| N-Methyl-N-nitrosourea | — | — | 217.842 | 303.869 | 244.714 |
| N-Methyl-N′-nitro-N- | — | — | 214.854 | — | — |
| nitrosoguanidine | | | | | |
| Dichloromethane | 22105.058 | 23154.503 | 23670.421 | 34170.726 | 43995.528 |
| N-Ethyl-N-nitrosourea | 312.549 | 258.996 | 277.036 | 315.767 | 368.165 |
| 2-Amino-3- | 534.016 | 507.707 | 541.138 | 406.002 | 415.772 |
| methylimidazo[4,5- | | | | | |
| f]quinoline | | | | | |
| Dimethyl sulfate | 289.582 | 319.472 | 369.481 | 439.424 | 397.003 |
| Acrylamide | 475.664 | 221.204 | 190.232 | 237.405 | 223.741 |
| Dimethylcarbamoyl chloride | — | — | 163.117 | 324.431 | 249.903 |
| 2-Nitrotoluene | 734.880 | 428.021 | 478.010 | 424.039 | 438.957 |
| 2-Nitroanisole | 333.279 | — | 223.279 | — | — |
| Styrene-7,8-oxide | 8838.281 | 5466.526 | 4208.660 | 4930.556 | 4508.500 |
| 1,2,3-Trichloropropane | — | 339.849 | — | — | 386.417 |

PFAS

The breath sample of two subjects was assayed for the presence of six Toxins. The breath sample assay was run using a high-resolution mass spectrometer coupled to a SUPER SESI™ device run on negative-ion mode. The numbers in the peak m/z column reflect the absolute value of a ratio of mass (i.e., Daltons) to charge at the center of the peak determined to correspond to the relevant compound. The numbers in the integrated IEC column reflect the area appearing under each respective peak on a produced extracted ion chromatogram, indicating relative abundance of the respective Toxin in the breath sample. A “—” denotes non-detection. Non-detection indicates that the respective compound was not present in the breath sample above the limit of detection. Most Toxins are detected at trace levels. Some Toxins are not detected in the breath sample assay, which may indicate a high elimination rate of the Toxin in the human body or a limited exposure of the subject to the Toxin. The results of the breath sample assay appear in Table 5 for Subject 1 and Table 2 for Subject 2, as can be seen below:

TABLE 5
Detection of Group Toxins in Subject 1

| | | | Integrated | |
|---|---|---|---|---|
| Candidate | | peak m/z | EIC | |
| compound | Formula | (center) | (normalized) | x1000 |
| PFHpA | C7HF13O2 | 362.969604 | 0.002340499 | 2.340498824 |
| PFHxA | C6HF11O2 | 312.974335 | 0.004738663 | 4.738662845 |
| PFHxS | C6HF13O3S | 398.937622 | 0.002722404 | 2.722404295 |
| PFPeA | C5HF9O2 | 262.977722 | 0.002445713 | 2.445712517 |
| PFPeS | C5HF11O3S | — | — | — |
| PFOA | C8HF15O2 | — | — | — |

TABLE 6
Detection of Group Toxins in Subject 2

| | | | Integrated | |
|---|---|---|---|---|
| Candidate | | peak m/z | EIC | |
| compound | Formula | (center) | (normalized) | x1000 |
| PFHpA | C7HF13O2 | 362.966858 | 0.001134247 | 1.134247416 |
| PFHxA | C6HF11O2 | 312.974854 | 0.000934994 | 0.934993525 |
| PFHxS | C6HF13O3S | 398.933044 | 0.000362942 | 0.362942033 |
| PFPeA | C5HF9O2 | 262.978363 | 0.000590049 | 0.590048618 |
| PFPeS | C5HF11O3S | — | — | — |
| PFOA | C8HF15O2 | 412.966583 | 5.52769E−05 | 0.055276886 |

TCE and Associated Byproducts

The breath sample of a healthy subject was assayed for the presence TCE and six TCE associated byproducts. The breath sample assay was run using a high-resolution mass spectrometer coupled to a SUPER SESI™ device run on negative-ion mode. The numbers reflect a value of abundance and a “—” denotes non-detection. Non-detection indicates that the respective compound was not present in the breath sample above the limit of detection. Most agents are detected at trace levels, some in only one or two of the five breaths assayed. Some agents are not detected in any breaths of the breath sample assay, which may indicate a high elimination rate of the agent in the human body or a limited exposure of the subject to the agent. The results of the breath sample assay appear in Table 7, below:

TABLE 7
Detection of TCE and associated byproducts

| Candidate compound | Breath 1 | Breath 2 | Breath 3 | Breath 4 | Breath 5 |
|---|---|---|---|---|---|
| trichloroethylene | 356.711 | 210.523 | — | — | — |
| trichloro acetic acid | — | — | — | — | — |
| dichloroacetic acid | 2314.812 | 2332.001 | 2038.649 | 1808.007 | 1662.866 |
| trichloroethanol | — | — | — | — | — |
| 1,2,dichlorovinyl glutathione | — | — | — | — | — |
| 1,2-dichlorovinyl cysteine | 247.679 | — | — | — | — |
| (DCVC) | | | | | |
| chlorothioketene | — | — | — | — | — |

II. Generating a Toxin Exposure Assessment

A toxin exposure assessment was generated from the results of a breath sample assay in accordance with embodiments of the invention.

FIGS. 6A-6B provide a depiction of the toxin exposure assessment obtained from the breath sample assay. In FIG. 6A, first page 600 of the toxin exposure assessment includes header 601 and selectable menu 602 for navigating between sections of the assessment when it is displayed on an electronic viewing device. Background section 604 is also provided along with session summary 603 providing information pertaining to the session in which the breath sample assay was performed. Table 605 summarizes the findings of the toxin exposure assessment, listing each selected toxin in a row with an assigned detection level reflecting a relative value of abundance for the toxin. In FIG. 6B, second page 606 of the toxin exposure assessment breaks each selected toxin into one of tables 607-609 based on a classification of each toxin. Each of tables 607-609 list selected toxins classified in the respective category in a row with the assigned detection level and a note highlighting any changes in detected toxin level from a previous breath sample of the subject. Chart 610 summarizes the results of the breath sample assay.

III. Generating a Health Evaluation

A health evaluation was generated based in part on the results of a toxin exposure assessment in accordance with embodiments of the invention.

FIG. 4 provides a depiction of the health evaluation obtained in part on the toxin exposure assessment. In FIG. 4, first page 400 of the health evaluation includes header 401 including information pertaining to the session in which the health evaluation was generated and identifying information of the subject. Diagnostics section 402 includes breath assay data 403 including a chart summarizing results of the toxin exposure assessment and a chart depicting compounds detected in the breath assay associated with various diseases or conditions. Interpretation section 404 explains the significance of the breath assay data on the subject's lung health and the health risks toxins may pose to the subject. Second page 405 of the health evaluation includes toxin health risk evolution 406 and various health scores 407 obtained as described above. Personal insights 408 are provided as charts depicting evolutions of the subject's overall health and lung health over the previous year and up to the present timepoint the depicted health evaluation was obtained.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C″ would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method comprising:
assaying a breath sample from a subject for the presence of one or more toxins to obtain a toxin exposure assessment for the subject; and
providing a health evaluation based on the toxin exposure assessment to the subject.

2. The method according to claim 1, wherein the one or more toxins includes one or more carcinogens.

3. The method according to claim 1, wherein the one or more toxins includes a toxin and/or metabolite thereof selected from the group consisting of Per- and Polyfluoroalkyl Substances (PFAS), Trichloroethylene, 1,3-Butadiene, Chlornaphazine, Thiotepa, Bis(chloromethyl) ether, Phenacetin, Benzene, Vinyl Chloride, 1,2-Dichloropropane, 2-Napthylamine, 4-Aminobiphenyl, Ortho Toluidine, Dichloromethane, N,N-Dimethylformamide and Styrene-7,8 oxide.

4-5. (canceled)

6. The method according to claim 1, wherein the health evaluation comprises a determination about the level of one or more toxins in breath relative to a baseline.

7-9. (canceled)

10. The method according to claim 1, wherein the health evaluation comprises an overall health score that is a composite of the toxin exposure assessment and one or more additional health assessments.

11. The method according to claim 10, wherein the one or more additional health assessments comprises a health assessment selected from the group consisting of a lung health assessment, an assessment of fitness for a given task(s), an ultrasound assessment, a biological sample assessment and combinations thereof.

12. The method according to claim 1, wherein the breath sample comprises from 1 to 5 exhaled breaths.

13. The method according to claim 1, wherein the assaying comprises mass spectrometry.

14. The method according to claim 13, wherein the mass spectrometry comprises high-resolution mass spectrometry.

15. The method according to claim 14, wherein the mass spectrometry comprises secondary electrospray ionization-high-resolution mass spectrometry.

16. The method according to claim 13, wherein the analyzing further comprises automatically configuring the mass spectrometry analyzer to perform selected ion monitoring based on real-time feedback of the measurements of the mass spectrometry analyzer.

17-18. (canceled)

19. The method according to claim 13, wherein the analyzing further comprises automatically switching the ionization agent of the mass spectrometry analyzer.

20-21. (canceled)

22. The method according to claim 13, wherein the analyzing further comprises automatically switching the polarity of the mass spectrometry analyzer.

23-24. (canceled)

25. The method according to claim 1, wherein the health evaluation is generated in real-time.

26. The method according to claim 13, wherein the toxin exposure assessment is generated for the subject using a machine learning model.

27. The method according to claim 26, wherein the method further comprises:
generating breath assay data for a plurality of subjects using the mass spectrometer;
obtaining toxin exposure data for each of the plurality of subjects;
training a machine learning model to identify one or more toxin fingerprints using the breath assay data and the obtained toxin exposure data; and
applying the trained machine learning model to breath assay data, different from the breath assay data used to train the model, to generate a toxin exposure assessment regarding the toxin exposure for a subject or subjects.

28. The method according to claim 1, wherein the method further comprises providing a therapy recommendation to the subject based on the health evaluation.

29. The method according to claim 28, wherein the therapy recommendation comprises a detoxication regimen.

30. The method according to claim 29, wherein the method further comprises administering the detoxification regimen to the subject.

31. The method according to claim 1, wherein the subject is a human.

32. The method according to claim 31, wherein the human is a protective service professional, a healthcare professional, a construction professional, a production professional or a military professional.

33. The method according to claim 32, wherein the human is a protective service professional.

34. The method according to claim 33, wherein the human is a fire fighter.

35-62. (canceled)
